(19) United States
(12) Patent Application Publication
(10) Pub. No.: US 2023/0310444 A1
(43) Pub. Date: Oct. 5, 2023

(54) COMPOSITIONS AND METHODS FOR TREATING DISEASES AND DISORDERS

(71) Applicant: Curis, Inc., Lexington, MA (US)

(72) Inventors: Reinhard von Roemeling, Ridgefield, CT (US); Elizabeth Martinez, Durham, NC (US); Robert Martell, Weston, MA (US)

(21) Appl. No.: 18/019,400

(22) PCT Filed: Apr. 30, 2021
(86) PCT No.: PCT/US2021/030192

§ 371 Date: Feb. 2, 2023

Related U.S. Application Data
(60) Provisional application No. 63/115,312, filed on Nov. 18, 2020.
(60) Provisional application No. 63/060,413, filed on Aug. 3, 2020.

(51) Int. Cl. A61K 31/519; A61K 31/4985; A61K 9/20; A61K 31/5377

(52) U.S. Cl. CPC A61K 31/5377; A61K 9/2018; A61K 9/2054; A61K 31/4985; A61K 31/519

(57) ABSTRACT

The present disclosure relates to methods of treating certain diseases and disorders (e.g., IRAK4 associated diseases and disorders). The present disclosure also relates to pharmaceutical compositions comprising compounds for treating the aforementioned diseases and disorders.

### RELATED APPLICATIONS

This application claims the benefit of U.S. Provisional Pat. Application No.: 63/115,312, filed on Nov. 18, 2020 and U.S. Provisional Pat. Application No.: 63/060,413, filed Aug. 3, 2020, the contents of each of which are hereby incorporated by reference in their entirety.

### BACKGROUND

Interleukin-1 (IL-1) Receptor-Associated Kinase 4 (IRAK4) is a serine/threonine kinase enzyme that plays an essential role in signal transduction by Toll/IL-1 receptors (TIRs). Diverse IRAK enzymes are key components in the signal transduction pathways mediated by interleukin-1 receptor (IL-1R) and Toll-like receptors (TLRs) (Janssens, S, et al. Mol. Cell. 11, 2003, 293-302). There are four members in the mammalian IRAK family: IRAK-1, IRAK-2, IRAK-M and IRAK4. These proteins are characterized by a typical N-terminal death domain that mediates interaction with MyD88-family adaptor proteins and a centrally located kinase domain. The IRAK proteins, as well as MyD88, have been shown to play a role in transducing signals other than those originating from IL-1R receptors, including signals triggered by activation of IL-18 receptors (Kanakaraj, et al. J. Exp. Med. 189(7):1999, 1129-38) and LPS receptors (Yang, et al., J. Immunol. 163, 1999, 639-643). Out of four members in the mammalian IRAK family, IRAK4 is considered to be the “master IRAK”. Under overexpression conditions, all IRAKs can mediate the activation of nuclear factor-kB (NF-kB) and stress-induced mitogen activated protein kinase (MAPK)-signaling cascades. However, only IRAK-1 and IRAK4 have been shown to have active kinase activity. While IRAK-1 kinase activity could be dispensable for its function in IL-1-induced NF-kB activation (Kanakaraj et al, J. Exp. Med. 187(12), 1998, 2073-2079) and (Xiaoxia Li, et al. Mol. Cell. Biol. 19(7), 1999, 4643-4652), IRAK4 requires its kinase activity for signal transduction (Li S, et al. Proc. Natl. Acad. Sci. USA 99(8), 2002, 5567-5572) and (Lye, E et al, J. Biol. Chem. 279(39); 2004, 40653-8). Given the central role of IRAK4 in Toll-like/IL-1R signalling and immunological protection, IRAK4 inhibitors have been implicated as valuable therapeutics in inflammatory diseases, sepsis and autoimmune disorders (Wietek C, et al, Mol. Interv. 2: 2002, 212-215).

Mice lacking IRAK4 are viable and show complete abrogation of inflammatory cytokine production in response to IL-1, IL-18 or LPS (Suzuki et al. Nature, 416(6882), 2002, 750-756). Similarly, human patients lacking IRAK4 are severely immune-compromised and are not responsive to these cytokines (Medvedev et al. J. Exp. Med., 198(4), 2003, 521-531 and Picard et al. Science 299(5615), 2003, 2076-2079). Knock-in mice containing inactive IRAK4 were completely resistant to lipopolysaccharide- and CpG-induced shock ( Kim TW, et al. J Exp Med 204: 2007, 1025 -36) and (Kawagoe T, et al. J Exp Med 204(5): 2007, 1013-1024) and illustrated that IRAK4 kinase activity is essential for cytokine production, activation of MAPKs and induction of NF- κ B regulated genes in response to TLR ligands (Koziczak-Holbro M, et al. J Biol Chem; 282(18): 2007;13552-13560). Inactivation of IRAK4 kinase (IRAK4 KI) in mice leads to resistance to EAE due to reduction in infiltrating inflammatory cells into CNS and reduced antigen specific CD4+ T-cell mediated IL-17 production (Kirk A et al. The Journal of Immunology, 183(1), 2009, 568-577).

Non-Hodgkin lymphoma (NHL) is the most common hematologic malignancy in adults with approximately 78 thousand new cases and 20 thousand deaths estimated for 2020 in the United States. The molecular pathology driving NHL is varied, although a common theme is over activity of the NFkB signaling pathway. Specific molecular changes have been identified that drive this pathway is subsets of NHL. For example, Diffuse large B-cell lymphoma (hereafter also referred to as “DLBCL”) is an aggressive lymphoma that can arise in lymph nodes or outside of the lymphatic system, in the gastrointestinal tract, testes, thyroid, skin, breast, bone, or brain. DLBCL is a cancer of B cells, a type of white blood cell responsible for producing antibodies. It is the most common type of non-Hodgkin’s lymphoma among adults, with an annual incidence of 7-8 cases per 100,000 people per year. This cancer occurs primarily in older individuals, with a median age of diagnosis at approximately 70 years of age, though it can also occur in children and young adults in rare cases. DLBCL is an aggressive tumor and the first sign of this illness is typically the observation of a rapidly growing mass. The five-year survival rate is only 58%.DLBCL has subtypes that are named according to their cell of origin and include germinal center B-cell-like (GCB) and activated B-cell-like (ABC). They differ in having a worse prognosis and, in some cases, requiring particularized approaches to treatment.

Another example of a NHL is Waldenstrom’s macroglobulinemia (WM). WM is a non-Hodgkin’s lymphoma that affects two types of B cells, lymphoplasmacytoid cells and plasma cells. WM is characterized by having high levels of a circulating antibody, immunoglobulin M (IgM), which is made and secreted by the cells involved in the disease. WM is a rare disease, with only about 1,500 cases per year in the United States. There is no single accepted treatment for WM and a marked variation in clinical outcome due to gaps in knowledge of the disease’s molecular basis. Objective response rates are high (> 80%) but complete response rates are low (0-15%).

Other types of non-Hodgkin’s lymphoma include mantle cell lymphoma (MCL), marginal zone lymphoma (MZL), follicular lymphoma (FL), chronic lymphocytic leukemia (CLL), small lymphocytic lymphoma (SLL), CNS lymphoma, and testicular lymphoma. Non-Hodgkin’s lymphoma can be caused by a variety of factors such as infections agents (Epstein-Barr virus, hepatitis C virus and human T-Cell leukemia virus), radiation and chemotherapy treatments, and autoimmune diseases. As a group, non-Hodgkin’s lymphoma affects 2.1% of the US population during their life. The percentage of people who survive beyond five years after diagnosis is 71%. In view of the foregoing, there is a clear and unmet need for additional therapies for the treatment of cancers and other diseases associated with IRAK4.

### SUMMARY

In one aspect, the present disclosure provides methods of treating disease or disorder in a subject in need thereof, comprising administering 50 - 500 mg of a compound to the subject, wherein the compound is

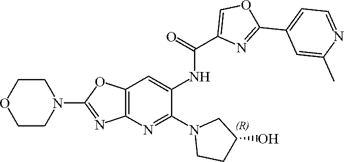 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In another aspect, the present disclosure provides methods of treating diseases or disorders associated with IRAK4 in a subject in need thereof, comprising administering 50 - 500 mg of a compound to the subject, wherein the compound is

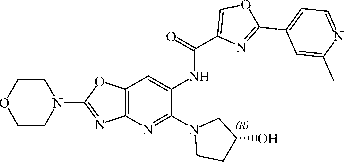 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides methods of treating a disease or disorder in a subject in need thereof, comprising conjointly administering 25 - 500 mg of a compound and a BTK inhibitor or a BCL-2 inhibitor to the subject, wherein the compound is

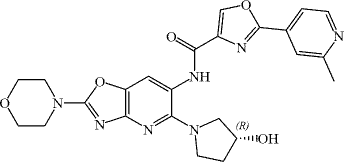 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides methods of treating a disease or disorder associated with IRAK4 in a subject in need thereof, comprising administering 25 - 500 mg of a compound and a BTK inhibitor or a BCL-2 inhibitor to the subject, wherein the compound is

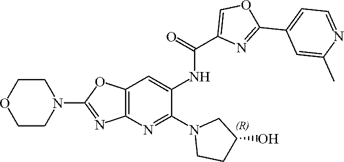 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides pharmaceutical compositions comprising 50 - 500 mg of compound and a pharmaceutically acceptable excipient, wherein the compound is

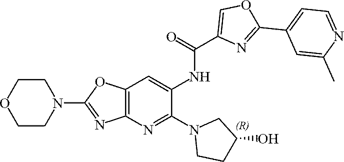 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides a unit dosage form comprising the pharmaceutical composition of the disclosure.

### BRIEF DESCRIPTION OF THE DRAWINGS

FIG. 1 shows a dose-dependent objective response to a human patient receiving Compound 1 at certain dosages.

FIG. 2 is a schematic of the IRAK¼ Complex with adapter protein MYD88. After substrate binding to IL-R1 or TLR, MYD88 activation recruits IRAK4/1 complex allowing IRAK-1 phosphorylation. Phosphorylated IRAK-1 then binds to TRAF-6 activating NK-kB signaling causing inflammation and tumor promotion. MYD88-L265P mutation leads to sustained upregulation of this pathway. Compound 1 inhibits IRAK4.

FIG. 3 shows a dose-dependent objective response to a human patient receiving Compound 1 at certain dosages.

FIG. 4 shows the response of certain human patients who received Compound 1.

FIGS. 5A-C show the efficacy of Compound 1 against certain in vivo models of non-Hodgkin’s lymphoma. In each instance, administration of Compound 1 reduced tumor growth.

FIG. 6 shows the efficacy of Compound 1 in combination with ibrutinib. The combination of Compound 1 and ibrutinib demonstrated synergistic reduction of tumor growth as compared to either Compound 1 or ibrutinib alone.

FIG. 7 shows the oral pharmacological profile of exemplary dosages of Compound 1. After oral administration, Compound 1 is rapidly absorbed with maximum plasma concentrations observed at 0.5-8 hours post dose. Compound 1 exhibits dose-proportional increase in exposure and has a half-life of approximately 6 hours. Minimal to no accumulation is observed following multiple daily single dose administration. Moderate accumulation is observed at steady state following multiple daily twice dose administration. In summary, the oral pharmacokinetics of Compound 1 are desirable.

FIG. 8 shows the percentage reduction in tumor burden for subjects who received 300 mg BID. Compound 1 has an acceptable safety and tolerability profile at RP2D, including 3 patients who have been on the study 1-2 years.

FIG. 9A shows the effects of exemplary concentrations of Compound 1 on erythroid differentiation from Primary MDS/AML Hematopoietic Stem and Progenitor Cells (HSCPs).

FIG. 9B shows the effects of exemplary concentrations of Compound 1 on neutrophilic differentiation from Primary MDS/AML Hematopoietic Stem and Progenitor Cells (HSCPs).

FIG. 10A shows the effects of Compound 1 on spleen weight in leukemic xerographs following 6 weeks of treatment at 12.5 mg/kg.

FIG. 10B shows the effects of Compound 1 on liver weight in leukemic xerographs following 6 weeks of treatment at 12.5 mg/kg.

FIG. 10C shows the effects of Compound 1 on the % of leukemic cells in the bone marrow in leukemic xerographs following 6 weeks of treatment at 12.5 mg/kg. Compound 1 decreased the disease burden in THP-1 xerographs.

FIG. 11 illustrates the design of the study described in Example 4.

### DETAILED DESCRIPTION

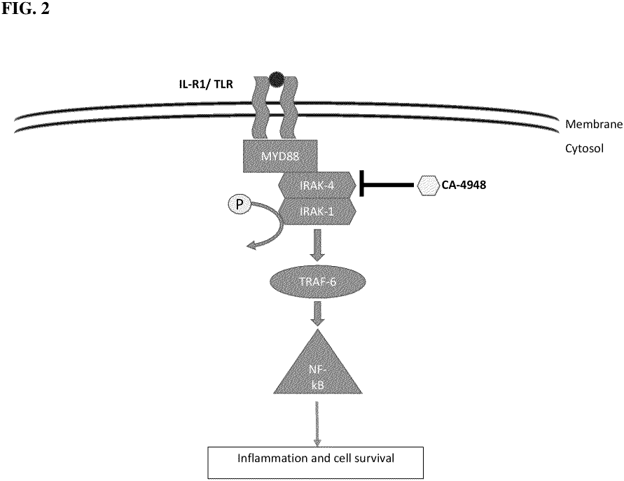

IL-1R associated kinase 4 (IRAK4) is a serine/threonine kinase that is a key component of the myddosome complex. The myddosome, a key component of the innate immune system, transmits intracellular signals from IL-1R and Toll-Like Receptors (TLR), resulting in NFkB activation. This pathway is also oncogenic in a number of malignancies. In greater than 90% of WM cases, a common activating missense mutation in the adapter protein Myeloid Differentiation Primary Response 88 (MYD88), occurs in which leucine is substituted for proline at position 265 (L265P) leading to uncontrolled proliferation. MYD88 is composed of a death domain at the N terminus, an intermediate linker domain, and a Toll/interleukin-1 receptor domain at the C terminus (FIG. 2). Toll-like receptor (TLR) and the interleukin 1 family of receptors (IL-1R) signaling occurs through MYD88. MYD88 is involved in the assembly and activation of IL-1R associated kinase 4 (IRAK4) which stimulates NF-kB mediated anti-apoptotic signaling cascades. IL-1R associated kinase 1 (IRAK1) is recruited by MYD88 via direct interaction with IRAK4, the most proximal IRAK. IRAK1 activation ultimately results in formation of the TRAF6-TAK1-IKK (tumor necrosis factor receptor associated factor 6- transforming growth factor beta-activated kinase 1-IkB kinase), activation of the NF-kB pathway, and promotion of cell survival.

Oncogenic signaling is transmitted by the myddosome, which requires IRAK4 for activation. TLRs are widely expressed on tumor cells, regulating growth and other tumor functions . MYD88 is a known oncogene which is mutated in a number of malignancies and requires IRAK4. The long form of IRAK4 (IRAK4-L) is itself known to be oncogenic in AML and MDS, where over half of cases overexpress IRAK4-L. Compound 1 is the first clinical candidate targeting IRAK4 to be evaluated in cancer patients and disclosed herein is evidence that targeting IRAK4 results in anti-cancer activity in a patient.

In one aspect, the present disclosure provides methods of treating disease or disorder in a subject in need thereof, comprising administering 50 - 500 mg of a compound to the subject, wherein the compound is

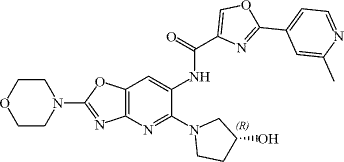 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In another aspect, the present disclosure provides methods of treating diseases or disorders associated with IRAK4 in a subject in need thereof, comprising administering 50 - 500 mg of a compound to the subject, wherein the compound is

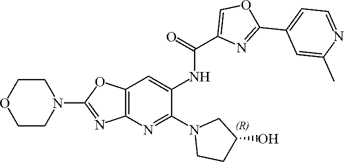 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides methods of treating a disease or disorder in a subject in need thereof, comprising conjointly administering 25 - 500 mg of a compound and a BTK inhibitor or a BCL-2 inhibitor to the subject, wherein the compound is

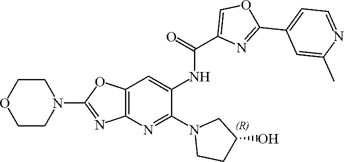 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof.

In yet another aspect, the present disclosure provides methods of treating a disease or disorder associated with IRAK4 in a subject in need thereof, comprising administering 25 - 500 mg of a compound and a BTK inhibitor or a BCL-2 inhibitor to the subject, wherein the compound

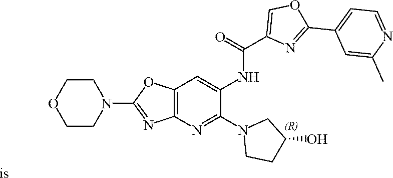

(i.e., Compound 1) or a pharmaceutically acceptable salt thereof.

In certain embodiments of the methods disclosed herein, the method comprises conjointly administering 25 - 500 mg of a compound and BCL-2 inhibitor to the subject, wherein the compound is

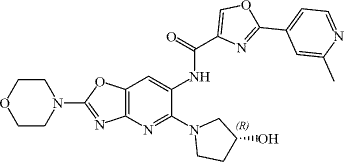 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain preferred embodiments, the BCL-2 inhibitor is ventoclax. In certain embodiments, the method comprises administering 400 mg of venetoclax daily. In certain embodiments, the ventoclax is administered orally. In certain preferred embodiments, the method comprises orally administering 400 mg of venetoclax daily. In other embodiments, the method comprises conjointly administering 25 - 500 mg of a compound and BTK inhibitor to the subject, wherein the compound is

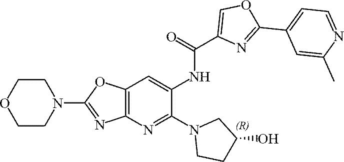 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the BTK inhibitor is ibrutinib, acalabrutinib, zanubrutinib, evobrutinib, ONO-4059, spebrutinib, or HM7 1224. In certain embodiments, the BTK inhibitor is acalabrutinib. In certain embodiments, the method comprises administering 200 mg of acalabrutinib daily. In certain embodiments, the acalabrutinib is administered orally. In certain embodiments, the method comprises orally administering 200 mg of acalabrutinib daily. In certain preferred embodiments, the BTK inhibitor is ibrutinib. In certain embodiments, the method comprises comprising administering 420 mg of ibrutinib daily. In other embodiments, the method comprises comprising administering 420 mg of ibrutinib daily. In certain embodiments, the ibrutinib is administered orally. In certain preferred embodiments, orally administering 420 mg of ibrutinib daily. In other preferred embodiments, the method comprisesadministering 560 mg of ibrutinib daily. In certain embodiments, the BTK inhibitor is zanubrutinib. In certain embodiments, the method administering 160 mg of zanubrutinib twice daily. In other embodiments, the method comprises administering 320 mg of zanubrutinib once daily. In certain embodiments, the zanubrutinib is administered orally. In certain embodiments, the method comprises orally administering 160 mg of zanubrutinib twice daily. In other embodiments, the method comprises orally administering 320 mg of zanubrutinib once daily.

Compound 1 may be administered in any amount or manner that elicits the desired response in the subject. For example, 100 - 400 mg of the compound can be administered to the subject twice per day or 200 - 1000 mg of the compound can be administered to the subject once per day. In certain embodiments, 100 - 400 mg of the compound is administered to the subject twice per day. In certain embodiments, 200 - 400 mg of the compound is administered to the subject twice per day. In certain preferred embodiments, 250 - 350 mg of the compound is administered to the subject twice per day. In certain embodiments, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound is administered to the subject twice per day. In certain embodiments, about 50 mg, about 75 mg, about 100 mg, about 200 mg, about 225 mg, about 250 mg, about 275 mg, about 300 mg, about 325 mg, about 350 mg, about 375 mg, or about 400 mg of the compound is administered to the subject twice per day. In certain embodiments, about 50 mg, about 100 mg, about 200 mg, or about 300 mg of the compound is administered to the subject twice per day. In certain embodiments, about 50 mg of the compound is administered to the subject twice per day. In other embodiments, about 200 mg of the compound is administered to the subject twice per day. In other embodiments, about 225 mg of the compound is administered to the subject twice per day. In other embodiments, about 250 mg of the compound is administered to the subject twice per day. In other embodiments, about 275 mg of the compound is administered to the subject twice per day. In particularly preferred embodiments, about 300 mg of the compound is administered to the subject twice per day. In other embodiments, about 325 mg of the compound is administered to the subject twice per day. In other embodiments, about 350 mg of the compound is administered to the subject twice per day. In other embodiments, about 375 mg of the compound is administered to the subject twice per day. In other embodiments, about 400 mg of the compound is administered to the subject twice per day.

In certain embodiments, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound to the subject once per day. In certain embodiments, about 50 mg of the compound to the subject once per day. In certain embodiments, about 75 mg of the compound to the subject once per day. In certain embodiments, about 100 mg of the compound to the subject once per day. In certain embodiments, about 125 mg of the compound to the subject once per day. In certain embodiments, about 150 mg of the compound to the subject once per day.

In certain preferred embodiments, the compound is orally administered to the subject. In certain embodiments, about 50 mg of the compound is orally administered to the subject twice per day. In other embodiments, about 200 mg of the compound is orally administered to the subject twice per day. In other embodiments, about 250 mg of the compound is orally administered to the subject twice per day. In particularly preferred embodiments, about 300 mg of the compound is orally administered to the subject twice per day. In other embodiments, about 325 mg of the compound is orally administered to the subject twice per day. In other embodiments, about 350 mg of the compound is orally administered to the subject twice per day. In other embodiments, about 375 mg of the compound is orally administered to the subject twice per day. In other embodiments, about 400 mg of the compound is orally administered to the subject twice per day. In other embodiments, about 50 mg of the compound to the subject once per day. In yet other embodiments, about 75 mg of the compound to the subject once per day. In yet other embodiments, about 100 mg of the compound to the subject once per day. In yet other embodiments, about 125 mg of the compound to the subject once per day. In yet other embodiments, about 150 mg of the compound to the subject once per day.

In certain embodiments, Compound 1 is administered continuously (e.g., Compound 1 is administered without a drug holiday). In other embodiments, Compound 1 is administered intermittently (e.g., Compound 1 is administered continuously interrupted by one or more drug holidays). In certain embodiments, each drug holiday lasts for a period of 1, 2, 3, 4, 5, 6, 7, 8, 9, 10, 11, 12, 13, or 14 days. In certain preferred embodiments, a drug holiday lasts for 7 days. In further preferred embodiments, Compound 1 is administered daily for three weeks followed by a one-week drug holiday, optionally followed by three weeks of daily administration and a one-week drug holiday, which cycle may be further repeated. In certain embodiments, the aforementioned dosing regimen continues, alternating periods of administration with holidays, until a change of disease state is observed (e.g., until a complete response, a partial response, or unacceptable toxicity is observed). Compound 1 can be administered to treat many diseases and disorders. For example, Compound 1 may be administered to treat diseases and disorders related to IRAK4. In certain embodiments, the disease or disorder is a cancer, preferably a hematological malignancy, such as a leukemia or lymphoma, for example a non-Hodgkin’s lymphoma. In certain embodiments, the hematological malignancy is myelogenous leukemia, myeloid leukemia (e.g., acute myeloid leukemia), myelodysplastic syndrome, lymphoblastic leukemia (e.g., acute lymphoblastic leukemia), chronic lymphocytic leukemia (CLL), small lymphocytic lymphoma (SLL), high risk CLL, follicular lymphoma, diffuse large B-cell lymphoma (DLBCL) (e.g., DLBCL or ABC-DLBLC), mantle cell lymphoma (MCL), Waldenstrom’s macroglobulinemia (WM), multiple myeloma, marginal zone lymphoma (MZL), Burkitt’s lymphoma, non-Burkitt high grade B cell lymphoma, extranodal marginal zone B cell lymphoma, transformed high grade B-cell lymphoma (HGBL), lymphoplasmacytic lymphoma (LPL), central nervous system lymphoma (CNSL), or MALT lymphoma. In certain embodiments, the hematological malignancy is myelogenous leukemia. In other embodiments, the hematological malignancy is myeloid leukemia (e.g., acute myeloid leukemia). In certain embodiments, the hematological malignancy is acute myeloid leukemia (e.g., AML). In certain embodiments, the AML is primary AML. In other embodiments, the AML is secondary AML. In yet other embodiments, the hematological malignancy is myelodysplastic syndrome. In certain embodiments, the myelodysplastic syndrome is high grade. In other embodiments, the myelodysplastic syndrome is low grade. In certain embodiments, the myelodysplastic syndrome is high risk. In yet other embodiments, the hematological malignancy is lymphoblastic leukemia (e.g., acute lymphoblastic leukemia). In yet other embodiments, the hematological malignancy is chronic lymphocytic leukemia (CLL). In certain embodiments, the CLL is high risk CLL. In yet other embodiments, the hematological malignancy is small lymphocytic lymphoma (SLL). In yet other embodiments, the hematological malignancy is follicular lymphoma. In yet other embodiments, the hematological malignancy is diffuse large B-cell lymphoma (DLBCL). In yet other embodiments, the hematological malignancy is activated B cell-like (ABC) DLBCL. In yet other embodiments, the hematological malignancy is germinal center B cell-like (GCB) DLBCL. In certain embodiments, the DLBCL is extranodal. In certain embodiments, the DLBCL is extranodal leg lymphoma, extranodal testicle lymphoma, or extra nodal not otherwise specified (NOS) type lymphoma. In yet other embodiments, the hematological malignancy is mantle cell lymphoma. In further embodiments, the hematological malignancy is Waldenstrom’s macroglobulinemia. In yet other embodiments, the hematological malignancy is multiple myeloma. In still other embodiments, the hematological malignancy is marginal zone lymphoma. In yet other embodiments, the hematological malignancy is Burkitt’s lymphoma. In yet other embodiments, the hematological malignancy is non-Burkitt high grade B cell lymphoma. In still other embodiments, the hematological malignancy is extranodal marginal zone B cell lymphoma. In yet other embodiments, the hematological malignancy is transformed high grade B-cell lymphoma (HGBL). In yet other embodiments, the hematological malignancy is lymphoplasmacytic lymphoma (LPL). In yet other embodiments, the hematological malignancy is CNS lymphoma. In yet other embodiments, the CNS lymphoma is primary CNS lymphoma (PCNSL). In yet other embodiments, the hematological malignancy is MALT lymphoma. In certain embodiments, the hematological malignancies described above may be relapsed or refractory. In certain embodiments, the hematological malignancies described above are resistant to treatment with a BTK inhibitor. In certain embodiments, the hematological malignancies described above are resistant to treatment with a BTK inhibitor as a monotherapy. In certain embodiments, the hematological malignancies is resistant to treatment with ibrutinib, acalabrutinib, zanubrutinib, evobrutinib, ONO-4059, spebrutinib, or HM7 1224. In certain preferred embodiments, the hematological malignancy is resistant to treatment with ibrutinib.

In certain embodiments, the cancer is selected from brain cancer, kidney cancer, liver cancer, stomach cancer, penile cancer, vaginal cancer, ovarian cancer, gastric cancer, breast cancer, bladder cancer, colon cancer, prostate cancer, pancreatic cancer, lung cancer, cervical cancer, epidermal cancer, prostate cancer, head or neck cancer. In certain preferred embodiments, the cancer is pancreatic cancer. In other embodiments, the cancer is colon cancer. In certain embodiments, the cancer is a solid tumor. In various such embodiments, the cancer may be relapsed or refractory. In certain embodiments, the cancers described above are resistant to treatment with a BTK inhibitor. In certain embodiments, the cancers described above are resistant to treatment with a BTK inhibitor as a monotherapy. In certain embodiments, the cancers are resistant to treatment with ibrutinib, acalabrutinib, zanubrutinib, evobrutinib, ONO-4059, spebrutinib, or HM7 1224. In certain preferred embodiments, the cancer is resistant to treatment with ibrutinib.

In other embodiments, the disease or disorder is an inflammatory disease or disorder. In certain embodiments, the inflammatory disease or disorder is an autoimmune disease or disorder. In certain embodiments, the inflammatory disease or disorder is an ocular allergy, conjunctivitis, keratoconjunctivitis sicca, vernal conjunctivitis, allergic rhinitis, autoimmune hematological disorders, hemolytic anemia, aplastic anemia, pure red cell anemia, idiopathic thrombocytopenia, systemic lupus erythematosus, rheumatoid arthritis, polychondritis, scleroderma, Wegener granulamatosis, dermatomyositis, chronic active hepatitis, myasthenia gravis, Steven- Johnson syndrome, idiopathic sprue, autoimmune inflammatory bowel disease, ulcerative colitis, Crohn’s disease, irritable bowel syndrome, celiac disease, periodontitis, hyaline membrane disease, kidney disease, glomerular disease, alcoholic liver disease, multiple sclerosis, endocrine opthalmopathy, Grave’s disease, sarcoidosis, alveolitis, chronic hypersensitivity pneumonitis, primary biliary cirrhosis, uveitis (anterior or posterior), Sjogren’s syndrome, interstitial lung fibrosis, psoriatic arthritis, systemic juvenile idiopathic arthritis, nephritis, vasculitis, diverticulitis, interstitial cystitis, glomerulonephritis, idiopathic nephrotic syndrome, minimal change nephropathy, chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic 13rticarial dysplasia, Behcet’s disease, incontinentia pigmenti, Paget’s disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison’s disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, 14 rticaria, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, 14 rticarial, bullous pemphigoid, pemphigus vulgaris, pemphigus foliaceus, paraneoplastic pemphigus, epidermolysis bullosa acquisita, acute or chronic gout, chronic gouty arthritis, psoriasis, psoriatic arthritis, rheumatoid arthritis, Cryopyrin Associated Periodic Syndrome (CAPS) and osteoarthritis. In certain preferred embodiments, the inflammatory disease or disorder is hypercytokinemia. In certain embodiments, the hypercytokinemia is induced by an infectious agent. In certain embodiments, the infectious agent is a virus. In certain preferred embodiments, the virus is a coronavirus (e.g., COVID-19). In other embodiments, the infectious agent is a bacteria. In certain embodiments, the inflammatory disease or disorder is graft vs host disease (GVHD). In certain embodiments, the GVHD is chornic graft vs host disease (cGVHD). In certain embodiments, the GVHD is sclerodermatous GVHD, steroid resistant GVHD, cyclosporin-resistant GVHD, GVHD, oral GVHD, reticular oral GVHD, erosive GVHD, or ulcerative oral GVHD. In certain embodiments, the GVHD is sclerodermatous GVHD. In certain embodiments, the GVHD is oral GVHD. In certain embodiments, the GVHD is reticular oral GVHD. In certain embodiments, the GVHD is erosive GVHD. In certain embodiments, the GVHD is ulcerative oral GVHD. In certain embodiments, the GVHD is overlap chronic GVHD. In certain embodiments, the GVHD is classic chronic GVHD. In certain embodiments, the GVHD is steroid resistant GVHD. In certain embodiments, the GVHD is cyclosporin-resistant GVHD. In certain embodiments, the GVHD is refractory. In certain embodiments, the GVHD is relapsed.

In certain embodiments, the diseases or disorders described above are resistant to treatment with a BTK inhibitor alone. In certain embodiments, the diseases or disorders described above are resistant to treatment with a BTK inhibitor as a monotherapy. In certain embodiments, the diseases or disorders are resistant to treatment with ibrutinib, acalabrutinib, zanubrutinib, evobrutinib, ONO-4059, spebrutinib, or HM7 1224. In certain preferred embodiments, the disease or disorder is resistant to treatment with ibrutinib.

In certain embodiments, the disease or disorder is associated with chronic anemia. In certain embodiments, the disease or disorder is chronic anemia. In certain embodiments, the disease or disorder is associated with transfusion dependency.

In certain embodiments, the subject is an adult human.

Compound 1 may be used as a first line therapy or it may be administered to patients who have failed to achieve a response, either partial or full, using one or more previous anti-cancer therapies or anti-inflammatory therapies. In certain embodiments, the subject has previously received at least one anti-cancer therapy. In certain embodiments, the patient has previously received one anti-cancer therapy. In other embodiments, the patient has previously received two anti-cancer therapies. In yet other embodiments, the patient has previously received three anti-cancer therapies. In yet other embodiments, the patient has previously received four anti-cancer therapies. In yet other embodiments, the patient has previously received five anti-cancer therapies. In certain embodiments, the at least one anti-cancer therapy is selected from an anti-CD20 antibody, a nitrogen mustard, a steroid, a purine analog, a DNA a topoisomerase inhibitor, a DNA intercalator, a tubulin inhibitor, a BCL-2 inhibitor, a proteasome inhibitor, a toll-like receptor inhibitor, a kinase inhibitor, an SRC kinase inhibitor, a PI3K kinase inhibitor, BTK inhibitor, a glutaminase inhibitor, a PD-1 inhibitor, a PD-L1 inhibitor and a methylating agent; or a combination thereof. In certain embodiments, the anti-cancer therapy is selected from ibrutinib, rituximab, bendamustine, bortezomib, dexamethasone, chlorambucil, cladribine, cyclophosphamide, doxorubicin, vincristine, venetoclax, ifosfamide, prednisone, oprozomib, ixazomib, acalabrutinib, zanubrutinib, IMO-08400, idelalisib, umbrelasib, CB-839, fludarabine, and thalidomide; or a combination thereof. In certain embodiments, the anti-cancer therapy is ibrutinib. In certain embodiments, the anti-cancer therapy is ibrutinib and rituximab. In certain embodiments, the anti-cancer therapy is bendamustine. In certain embodiments, the anti-cancer therapy is bendamustine and rituximab. In certain embodiments, the anti-cancer therapy is bortezomib. In certain embodiments, the anti-cancer therapy is bortezomib and dexamethasone. In certain embodiments, the anti-cancer therapy is bortezomib and rituximab. In certain embodiments, the anti-cancer therapy is bortezomib, rituximab, and dexamethasone. In certain embodiments, chlorambucil. In certain embodiments, the anti-cancer therapy is cladribine. In certain embodiments, the anti-cancer therapy is cladribine and rituximab. In certain embodiments, the anti-cancer therapy is cyclophosphamide, doxorubicin, vincristine, prednisone, and rituximab (i.e., CHOP-R). In certain embodiments, the anti-cancer therapy is cyclophosphamide, prednisone, and rituximab (i.e., CPR). In certain embodiments, the anti-cancer therapy is fludarabine. In certain embodiments, the anti-cancer therapy is fludarabine and rituximab. In certain embodiments, the anti-cancer therapy is fludarabine, cyclophosphamide, and rituximab. In certain preferred embodiments, the anti-cancer therapy is rituximab. In certain preferred embodiments, the anti-cancer therapy comprises rituximab. In certain embodiments, the anti-cancer therapy is rituximab, cyclophosphamide, and dexamethasone (i.e., RCD). In certain embodiments, the anti-cancer therapy is thalidomide. In certain embodiments, the anti-cancer therapy is thalidomide and rituximab. In certain embodiments, the anti-cancer therapy is venetoclax. In certain embodiments, the anti-cancer therapy is cyclophosphamide, bortezomib, and dexamethasone (i.e. R-CyBorD). In certain embodiments, the anti-cancer therapy is a hypomethylating agent. In certain embodiments, the subject has previously received at least 6 cycles of a hypomethylating agent. In certain embodiments, the anti-cancer therapy is a combination of any of the foregoing, for example the subject may first receive rituximab and then at a later date receive a combination of rituximab, cyclophosphamide, and dexamethasone (i.e., RCD).

In certain embodiments, the subject has previously received at least one anti-inflammatory therapy. In certain embodiments, the patient has previously received one anti-inflammatory therapy. In other embodiments, the patient has previously received two anti-inflammatory therapies. In yet other embodiments, the patient has previously received three anti-inflammatory therapies. In yet other embodiments, the patient has previously received four anti-inflammatory therapies. In certain embodiments, the anti-inflammatory is a steroid (e.g., corticosteroid). In certain embodiments, the anti-inflammatory therapy is hydrocortisone, cortisone, ethamethasoneb, prednisone, prednisolone, triamcinolone, dexamethasone, or fludrocortisone; or a combination thereof.

The subject may also have received or been prepared for other, non-chemotherapeutic treatments, such as surgery, radiation, or a bone marrow transplant. In certain embodiments, the subject has previously received etoposide chemo-mobilization therapy. In certain embodiments, the subject has previously received a bone marrow transplant. In certain embodiments, he subject has previously received a stem cell transplant. In certain embodiments, the subject has previously received an autologous cell transplant. In certain embodiments, the subject has previously received an allogenic stem cell transplant. In certain embodiments, the subject has previously received a hematopoietic cell transplantation. In certain embodiments, the subject has previously received carmustine, etoposide, cytarabine, and melphalan (i.e., BEAM conditioning). In certain embodiments, the subject has previously received re-induction therapy.

The subject may have also previously exhibited a favorable outcome to prior therapy only to require additional treatment at a later date. In certain embodiments, the subject has previously achieved a partial response. In certain embodiments, the subject has previously achieved a good partial response. In certain embodiments, the subject has previously achieved a complete response. In certain embodiments, the cancer is relapsed. In certain embodiments, the cancer is refractory.

The subject may also have preexisting or developed one or more genetic mutations that render the subjects cancer more or less resistant to therapy. In certain embodiments, the subject has a mutation in RICTOR. In certain embodiments, the subject has a N1065S mutation in RICTOR. In certain preferred embodiments, the subject has a mutation in MYD88. In certain even further preferred embodiments, the subject has a L265P mutation in MYD88. In certain embodiments, the subject has a mutation in TET2. In certain embodiments, the subject does not have a mutation in CXCR4. In other embodiments, the subject has a mutation in CXCR4. In certain embodiments, the subject shows early progression. In certain embodiments, the subject has not previously received a BTK inhibitor.

In certain embodiments, following administration of the compound, the subject achieves a partial response. In certain embodiments, following administration of the compound, the subject achieves a good partial response. In other embodiments, following administration of the compound, the subject achieves a complete response. In certain embodiments, the subject achieves a partial response within 7 days of receiving the compound. In certain embodiments, the subject achieves a good partial response within 7 days of receiving the compound. In certain embodiments, the subject achieves a complete response within 7 days of receiving the compound. In certain embodiments, the subject’s tumor volume is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In certain embodiments, the subject’s tumor volume is reduced by 5%. In certain embodiments, the subject’s tumor volume is reduced by 10%. In certain embodiments, the subject’s tumor volume is reduced by 15%. In certain embodiments, the subject’s tumor volume is reduced by 20%. In certain embodiments, the subject’s tumor volume is reduced by 25%. In certain embodiments, the subject’s tumor volume is reduced by 30%. In certain embodiments, the subject’s tumor volume is reduced by 35%. In certain embodiments, the subject’s tumor volume is reduced by 40%. In certain embodiments, the subject’s tumor volume is reduced by 45%. In certain embodiments, the subject’s tumor volume is reduced by 50%. In certain embodiments, the subject’s tumor volume is reduced by 55%. In certain embodiments, the subject’s tumor volume is reduced by 60%. In certain embodiments, the subject’s tumor volume is reduced by 65%. In certain embodiments, the subject’s tumor volume is reduced by 70%. In certain embodiments, the subject’s tumor volume is reduced by 80%. In certain embodiments, the subject’s tumor volume is reduced by 85%. In certain embodiments, the subject’s tumor volume is reduced by 90%. In certain embodiments, the subject’s tumor volume is reduced by 95%.

In yet another aspect, the present disclosure provides methods of treating DLBCL in a subject in need thereof, comprising orally administering about 50 mg of a compound to the subject orally once per day, wherein the compound is

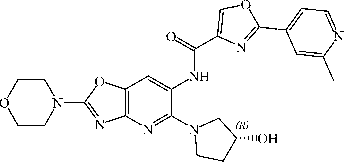 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the DLBCL is relapsed or refractory.

In yet another aspect, the present disclosure provides methods of treating FL in a subject in need thereof, comprising orally administering about 50 mg of a compound to the subject orally once per day, wherein the compound is

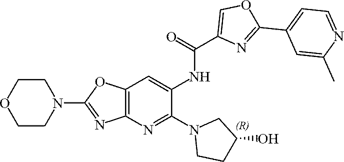 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the FL is relapsed or refractory.

In yet another aspect, the present disclosure provides methods of treating WM in a subject in need thereof, comprising orally administering about 300 mg of a compound to the subject orally twice per day, wherein the compound is

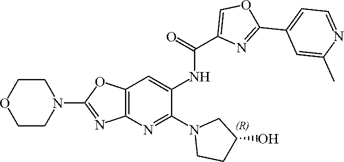 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the WM is relapsed or refractory.

In yet another aspect, the present disclosure provides methods of treating DLBCL in a subject in need thereof, comprising orally administering about 50 mg of a compound to the subject orally twice per day, wherein the compound is

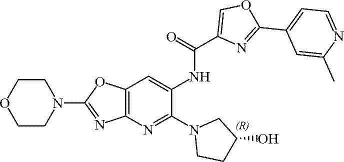 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the DLBCL is relapsed or refractory.

In yet another aspect, the present disclosure provides methods of treating LPL in a subject in need thereof, comprising orally administering about 300 mg of a compound to the subject orally twice per day, wherein the compound is

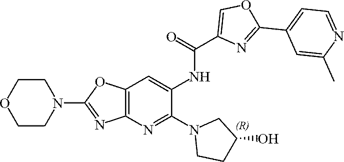 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the LPL is relapsed or refractory.

In yet another aspect, the present disclosure provides methods of treating GCB DLBCL in a subject in need thereof, comprising orally administering about 300 mg of a compound to the subject orally twice per day, wherein the compound is

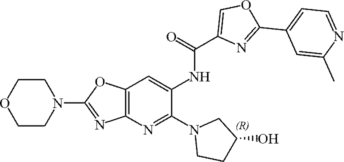 [SMILES: Cc1cc(-c2nc(C(=O)Nc3cc4oc(N5CCOCC5)nc4nc3N3CC[C@@H](O)C3)co2)ccn1]

(i.e.,Compound 1) or a pharmaceutically acceptable salt thereof. In certain embodiments, the GCB DLBCL is relapsed or refractory.

In yet another aspect, the present disclosure provides methods of treating ABC DLBCL in a subject in need thereof, comprising orally administering about 400 mg of a compound to the subject orally twice per day, wherein the compound is

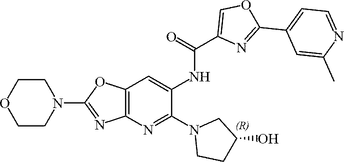 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the ABC DLBCL is relapsed or refractory.

In yet another aspect, the present disclosure provides methods of treating MZL in a subject in need thereof, comprising orally administering about 400 mg of a compound to the subject orally twice per day, wherein the compound is

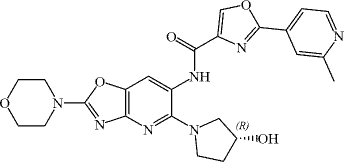 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the MZL is relapsed or refractory.

In yet another aspect, the present disclosure provides methods of treating MZL in a subject in need thereof, comprising orally administering about 300 mg of a compound to the subject orally twice per day, wherein the compound is

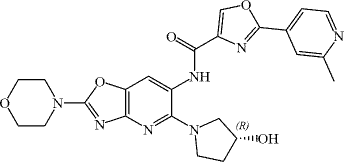 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the MZL is relapsed or refractory.

In yet another aspect, the present disclosure provides methods of treating MALT lymphoma in a subject in need thereof, comprising orally administering about 300 mg of a compound to the subject orally twice per day, wherein the compound is

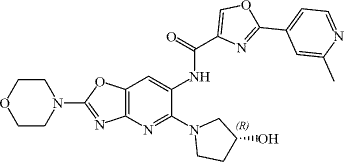 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, the MALT lymphoma is relapsed or refractory.

Upon receiving Compound 1, the subject may experience one or more additional benefits not directly associated with the treatment of the subjects cancer. For example, the subject may experience antineoplastic, immunomodulatory, and anti-inflammatory effects. In certain embodiments, following administration of the compound, the subjects IL-1 induced signaling decreases. In certain embodiments, following administration of the compound, the subjects cytokine production decreases. In certain embodiments, the subject is suffering from a secondary condition and the symptoms of the secondary condition are ameliorated. In certain embodiments, the subject is suffering from an autoimmune condition. In certain embodiments, following administration of the compound, the symptoms of the subjects autoimmune condition are ameliorated.

In yet another aspect, the present disclosure provides solid pharmaceutical compositions comprising the compound of the disclosure or a pharmaceutically acceptable salt thereof and a pharmaceutically acceptable excipient, such as a diluent. For example, in certain embodiments, the pharmaceutical composition comprises 50 - 500 mg of compound and a pharmaceutically acceptable excipient, wherein the compound is

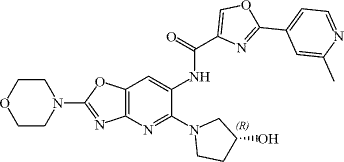 [SMILES: Cc1cc(-c2nc(C(=O)Nc3cc4oc(N5CCOCC5)nc4nc3N3CC[C@@H](O)C3)co2)ccn1]

(i.e., Compound 1) or a pharmaceutically acceptable salt thereof. In certain embodiments, thecompound is

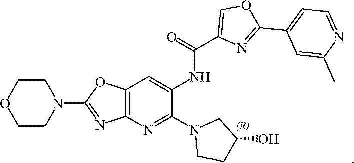

In other embodiments, the compound is a pharmaceutically acceptable salt of

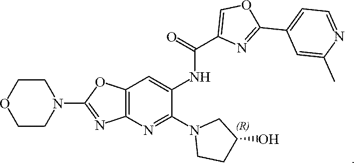

In certain embodiments, the pharmaceutical composition comprises 50 - 400 mg of the compound. In certain embodiments, the pharmaceutical composition comprises 100 - 400 mg of the compound. In certain embodiments, the pharmaceutical composition comprises 200 - 400 mg of the compound. In certain embodiments, the pharmaceutical composition comprises about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound. In certain embodiments, the pharmaceutical composition comprises about 50 mg, about 75 mg, about 100 mg, about 200 mg, about 225 mg, about 250 mg, about 275 mg, about 300 mg, about 325 mg, about 350 mg, about 375 mg, or about 400 mg of the compound. In certain embodiments, the pharmaceutical composition comprises about 50 mg, about 100 mg, about 200 mg, or about 300 mg of the compound. In certain embodiments, the pharmaceutical composition comprises about 50 mg of the compound. In certain embodiments, the pharmaceutical composition comprises about 75 mg of the compound. In certain embodiments, the pharmaceutical composition comprises about 100 mg of the compound. In certain embodiments, the pharmaceutical composition comprises about 125 mg of the compound. In certain embodiments, the pharmaceutical composition comprises about 150 mg of the compound. In certain embodiments, the pharmaceutical composition comprises about 200 mg of the compound. In certain embodiments, the pharmaceutical composition comprises about 225 mg of the compound. In certain embodiments, the pharmaceutical composition comprises about 250 mg of the compound. In certain embodiments, the pharmaceutical composition comprises about 275 mg of the compound. In certain preferred embodiments, the pharmaceutical composition comprises about 300 mg of the compound. In certain embodiments, the pharmaceutical composition comprises about 325 mg of the compound. In certain embodiments, the pharmaceutical composition comprises about 350 mg of the compound. In certain embodiments, the pharmaceutical composition comprises about 375 mg of the compound. In certain embodiments, the pharmaceutical composition comprises about 400 mg of the compound. In certain embodiments, the compound comprises about 5%, about 10%, or about 15% of the total weight of the pharmaceutical composition. In certain preferred embodiments, the compound comprises about 10% of the total weight of the pharmaceutical composition.

In certain embodiments, the pharmaceutically acceptable excipient is a diluent. In certain embodiments, the diluent comprises lactose monohydrate or microcrystalline cellulose; or a combination thereof. In certain preferred embodiments the diluent comprises lactose monohydrate and microcrystalline cellulose. In certain preferred embodiments, the diluent comprises about 75%, about 80%, or about 85% of the total weight of the pharmaceutical composition, most preferably about 80% of the total weight of the pharmaceutical composition.

In certain embodiments, the pharmaceutical composition further comprises a binder. In certain preferred embodiments, the binder is hypromellose. In certain preferred embodiments, the binder comprises about 2.5%, about 5%, or about 7.5% of the total weight of the pharmaceutical composition, most preferably about 5% of the total weight of the pharmaceutical composition.

In certain embodiments, the pharmaceutical composition further comprises a surfactant. In certain embodiments, the surfactant is a lauryl sulphate, preferably sodium lauryl sulphate. In certain preferred embodiments, the surfactant comprises about 0.5%, about 1%, or about 1.5% of the total weight of the pharmaceutical composition, most preferably about 1% of the total weight of the pharmaceutical composition.

In certain embodiments, the pharmaceutical composition further comprises a disintegrant. In certain embodiments, the disintegrant is a croscarmellose, preferably croscarmellose sodium. In certain preferred embodiments the disintegrant comprises about 1.5%, about 2%, or about 2.5% of the total weight of the pharmaceutical composition, most preferably about 1% of the total weight of the pharmaceutical composition.

In certain embodiments, the pharmaceutical composition further comprises a lubricant. In certain embodiments, the lubricant is a stearate, preferably magnesium stearate. In certain preferred embodiments, the lubricant comprises about 0.5%, about 1%, or about 1.5% of the total weight of the pharmaceutical composition, most preferably about 1% of the total weight of the pharmaceutical composition.

In certain embodiments, the pharmaceutical composition further comprises a solvent. In certain preferred embodiments, the solvent is water. In certain preferred embodiments, the solvent comprises less than 1% of the total weight of the pharmaceutical composition, preferably less than 0.5% or 0.1% of the pharmaceutical composition.

In certain embodiments, the pharmaceutical composition is in the form of a tablet or a capsule. In certain embodiments, the pharmaceutical composition is in the form of a tablet. In certain embodiments, the tablet is coated with polyvinyl alcohol, talc, titanium dioxide, non-irradiated yellow iron oxide, glyceryl monocaprylocaprate, or sodium lauryl sulphate; or a combination thereof. In certain embodiments, the tablet is coated with polyvinyl alcohol, talc, titanium dioxide, non-irradiated yellow iron oxide, glyceryl monocaprylocaprate, and sodium lauryl sulphate.

In yet another aspect, the present disclosure provides a unit dosage form comprising the pharmaceutical composition of the disclosure such that an integer number of unit dosage forms supplies an appropriate dose of the active ingredient (e.g., Compound 1), as disclosed elsewhere herein. In certain embodiments, 1, 2, 3, 4, 5, or 6 unit dosage forms supply a dose. In certain embodiments, one unit dosage form supplies the entire dose. In other embodiments, two unit dosage forms supply the entire dose. In yet other embodiments, three unit dosage forms supply the entire dose. In other embodiments, four unit dosage forms supply the entire dose. In other embodiments, five unit dosage forms supply the entire dose. In six embodiments, two unit dosage forms supply the entire dose.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient’s condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison’s Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetraalkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release. Furthermore, when certain amounts of a compound are specified (e.g., 500 mg of Compound 1 or a pharmaceutically acceptable salt thereof), the amount of the compound is calculated based on the free base of the compound without accounting for the additional weight of the salt counterion (e.g., acid of addition) that would increase the weight of the salt form of the compound. Thus, the same molar amount of the compound is indicated, whether the compound is included as the free base or a salt form.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject’s size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Compound 1. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Compound 1 are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Compound 1 for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Compound 1 or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Some of the compounds may also exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

The term “partial response” as used herein means an objective response in at least one organ or tissue in the subject with no evidence of progression elsewhere. For example, a partial response may refer to a 50% or more reduction in the disease state (e.g., tumor volume).

The term “complete response” as used herein means a complete disappearance of the measurable evidence of the disease in the subject. For example, in certain embodiments a complete response may refer to the complete measurable disappearance of the subject’s cancer. In other embodiments, a complete response may refer to the complete measurable disappearance of the subject’s symptoms (e.g., the subject’s cytokine count may return to normal).

### EXAMPLES

The invention now being generally described, it will be more readily understood by reference to the following examples which are included merely for purposes of illustration of certain aspects and embodiments of the present invention, and are not intended to limit the invention.

### Example 1: Performance of Compound 1 in WM

The patient is an otherwise healthy male who presented age 49 with complaints of severe fatigue. Routine labs were notable for an elevated erythrocyte sedimentation rate and anemia; therefore, he was referred to hematology/oncology. Further work-up revealed an IgM lambda m-protein on serum protein electrophoresis and a hypercellular bone marrow with trilineage hematopoiesis and an atypical lymphoplasmacytic infiltrate, consistent with WM. CT scans did not reveal lymphadenopathy or hepatosplenomegaly.

Due to symptomatic cytopenia and profound fatigue, treatment was recommended. The patient received rituximab induction 375 mg/m² IV weekly for 8 weeks then rituximab maintenance every 3 months for 8 doses between 2005 and 2007 achieving a very good partial remission. He did well for approximately 4 years at which time his disease progressed, and he developed recurrent symptomatic anemia as well as new grade 1 sensory peripheral neuropathy involving the hands and feet. He was retreated with rituximab between June and September of 2011 achieving stable disease with a numeric increase in IgM from 1476 to 2042 mg/dL during this time and no improvement in symptoms. Repeat bone marrow biopsy November 2011 showed a 90% cellular marrow with WM accounting for 20% of cellularity, IgM lambda plasma cells accounting for 5-10% of cellularity, normal cytogenetics, no increase in reticulin staining, and trace stainable iron. CT scans at that time were without lymphadenopathy or organomegaly. By December 2012, his serum IgM had increased to 3380 mg/dL, IgM lambda m-protein was 2.37 g/dL. He was referred to a tertiary care center for further management where induction chemotherapy followed by autologous stem cell transplantation was recommended. In early 2013 he received 2 cycles of rituximab, cyclophosphamide, bortezomib, and dexamethasone (R-CyBorD) achieving partial remission (PR) with a decrease in IgM to 1285 mg/dL and m-protein to 0.88 g/dL. The patient then received rituximab, ifosfamide, etoposide chemo-mobilization and stem cell collection in June 2013 after which his IgM and m-protein remained unchanged. Further cytoreduction with 2 cycles of bendamustine and rituximab (BR) was administered pre-transplant which deepened the patient’s partial response (IgM 454 mg/dL, m-protein 0.30 g.dL). He then underwent autologous stem cell transplantation with BEAM conditioning (carmustine, etoposide, cytarabine, melphalan) in October 2013 without complication and achieved a very good partial response (VGPR) with IgM nadir post-transplant of 135 mg/dL and m-protein detectable by immunofixation only in January 2014.

The patient remained asymptomatic for over 4 years with a slow increase in m-protein and IgM during that time. By mid-2017 he started to experience increased fatigue. Bone marrow evaluation 11/2017 revealed a normocellular marrow with 30% involvement by WM and normal cytogenetics. Next generation sequencing revealed RICTOR N1065S mutation as well as MYD88 L265P mutation and TET2 mutation in a suclonal population. There was no evidence of CXCR4 genomic alteration. By late 2018 his fatigue had started to interfere with his ability to perform his usual activities, so treatment was again recommended. Several options were discussed including clinical trials and standard of care Bruton’s tyrosine kinase inhibitor (BTKi) therapy. Given his clinical history, current symptoms, known mutational landscape, and personal preference he was enrolled in phase 1, dose-escalation study of the novel oral IRAK4 inhibitor, Compound 1, in patients with relapsed or refractory B-cell malignancies (NCT03328078).

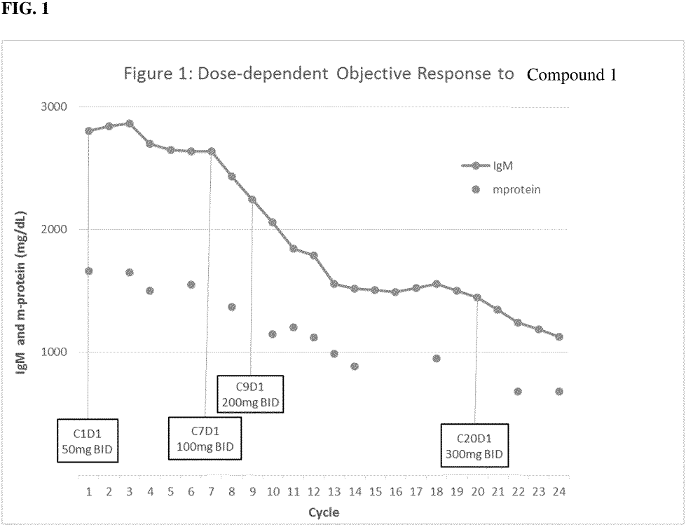

Baseline testing in December 2018 included a bone marrow biopsy showing 5-10% involvement by WM, m-protein of 1.66 g/dL, IgM 2,801 mg/dL, and a computed tomography scan without pathological lymphadenopathy or hepatosplenomegaly. Quantitative immunoglobulins and serum protein electrophoresis were obtained each cycle to determine response to treatment (FIG. 1).

The patient initiated treatment at the first dose level, 50 mg. He tolerated therapy well without adverse events. During the first six 21-day cycles his m-protein slowly but steadily trended down to 1.55 g/dL and IgM initially increased from 2801 to 2866 mg/dL during the first 2 cycles then decreased to 2639 by cycle 6 day 1 (FIG. 1). Using a standard 3+3 design the subsequent dose level, 100 mg po BID, was cleared per protocol. Considering evidence of response without noted toxicity, the patient was a candidate to escalate to 100 mg po BID starting with cycle 7 day 1 in April 2019. He had continued stable disease (SD) with down trending IgM and m-protein during cycles 7 and 8 without evidence of toxicity, and therefore became a candidate to escalate to the next cleared dose level of 200 mg po BID starting with cycle 9 day 1 in May 2019. Prior to 200 mg BID dose escalation his baseline IgM was 2245 mg/dL and m-protein of 1.37 g/dL. His baseline symptom of fatigue was slightly improved but persistent. He continued to enjoy a seemingly dose-dependent reduction in tumor markers at the 200 mg po BID dose level and by the summer of 2019 his fatigue had completely resolved. He reported a significant improvement in his quality of life with resolution of fatigue and returned to a rigorous daily exercise program that he had been unable to perform for the last 2 years due to WM-related symptoms. In August 2019 he was noted to have an asymptomatic grade 2 creatine phosphokinase (CPK) elevation, extensive work-up and physical exam was unrevealing. He reduced the intensity of his exercise program and the asymptomatic CPK elevation resolved completely without need for Compound 1 dose delay or reduction. During cycles 15 through 20, while on the 200 mg po BID dose, the patient’s IgM plateaued around 1500 mg/dL and m-protein plateaued near 0.9 g/dL. Based on evolving safety data at higher dose levels, the patient was escalated to 300 g po BID starting with cycle 20 in 1/2020. He again experienced a seeming dose-dependent acceleration of response without noted toxicity and achieved PR as of C22 day 1 (m-protein 0.68 g/dL, IgM 1241 mg/dL). He remains on Compound 1 300 mg po BID.

Therapies in WM involve targeting pathways associated with the known mutations of MYD88 and CXCR-4. Previous studies have shown the role of IRAK4 in signaling cascade involved in stimulatory effects of proinflammatory cytokines through forming a complex with MYD88. Thus, IRAK4 is an essential component in regulating immune responses and those with dysfunctions in either part of the complex can lead to immune deficiencies or immune dysregulation. With the addition of an IRAK4 inhibitor, a strong association is formed between IRAK4 and MYD88 and a weak association is formed with IRAK-1, thus reducing the ubiquitination of IRAK1 ultimately leading to decreased IL-1 induced signaling and cytokine production.

Through inhibition of IRAK4, Compound 1 prevents NF-kB activation, leading to decreased inflammatory cytokine production and potential antineoplastic, immunomodulatory, and anti-inflammatory effects. Preclinical studies also suggest that Compound 1 affects TLR/IL1R signaling which may prevent the inflammatory process in auto-immune conditions.

This patient has well tolerated continuous oral treatment with Compound 1 for close to 18 months. His tumor burden has been shrinking in a dose-dependent manner, reaching partial response status (PR) according to the 6^{th} International Workshop on WM response criteria (FIG. 1). The patient’s quality of life improved from baseline with resolution of fatigue, even allowing him vigorous physical exercise that he had enjoyed prior to disease onset. However, this resulted in intermittent asymptomatic grade 2 elevation of CK which resolved with exercise moderation without the need of holding or reducing Compound 1 exposure.

### Example 2: Performance of Compound 1 in DLBCL, FL, HGBL, WM, LPL, MZL, and MCL

### Study Design and Methods

Phase I trial Compound 1 is a dose escalation trial with a 3 + 3 design. Seven dosing cohorts included 50 and 100 mg QD, and 50, 100, 200, 300, or 400 mg BID of daily continuous oral monotherapy in 21-Day cycles. Objective included safety and tolerance (primary), pk/pd and early efficacy (secondary), and biomarker correlations (exploratory). 31 patients with resistant or refractory, advanced NHL have been enrolled. Details of the patient population are set forth in Table 1 below.

TABLE 1

Summary of Adverse Effects

| Characteristics & Disposition | Overall (N=31) |
|---|---|
| Male, n (%) | 26 (80) |
| Female, n (%) | 5 (16) |
| Age, median years (range) | 69 (40-75) |
| Histology, n (%) | |
| Diffuse large B-cell lymphoma (DLBCL) | 14 (45) |
| Transformed follicular lymphoma (t-FL/DLBCL) ^{∗} | 6 (19) |
| Waldenstrom’s Macroglobulinemia (WM) | 4 (13) |
| Other Lymphoma^{∗∗} | 4 (13) |
| No. prior regimens [Median (range)] | 4 (1-8) |
| Ibrutinib inhibitor, n (%) | 6 (19) |
| CAR-T, n (%) | 5 (16) |
| ASCT | 7(23) |

^{∗}High grade composite low-high grade disease per local pathology report
^{∗∗}Includes Lymphoplasmacytic (n=2), mantle cell (n=2), marginal zone (n=2), high grade MYC-BCL₆ (n-1

### Results

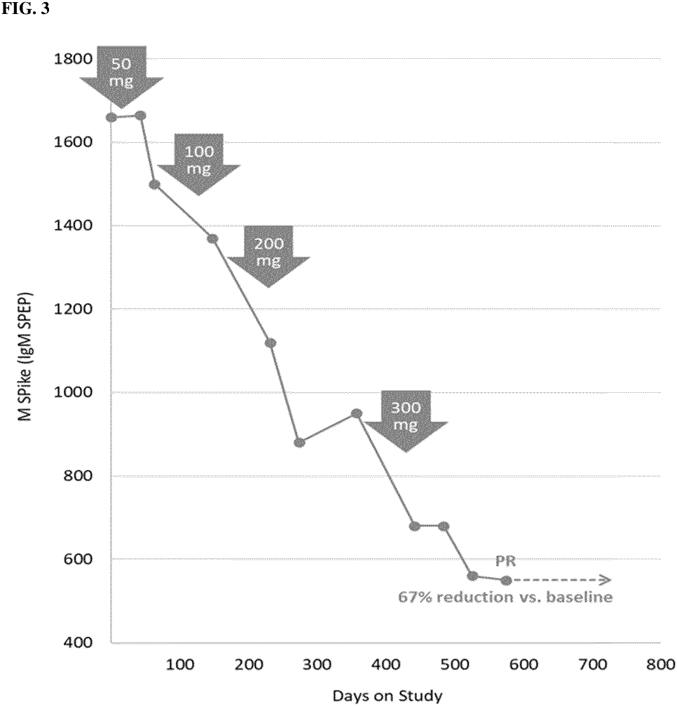

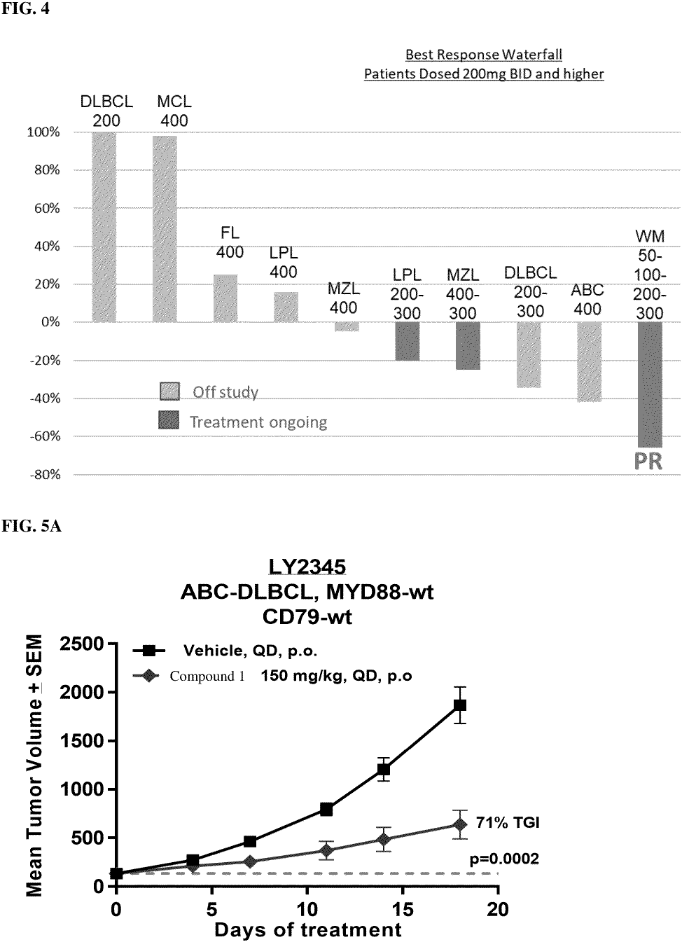

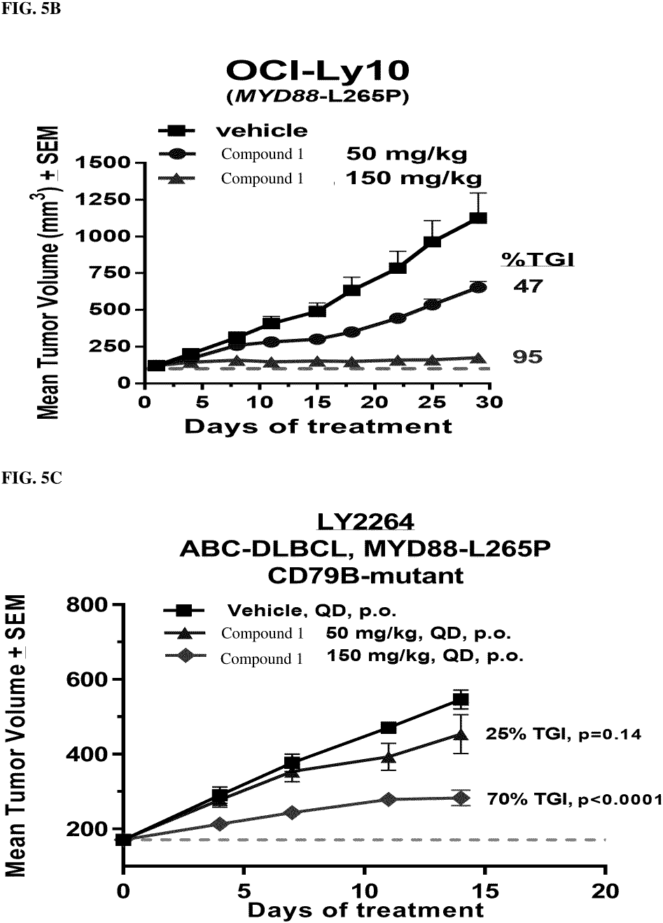

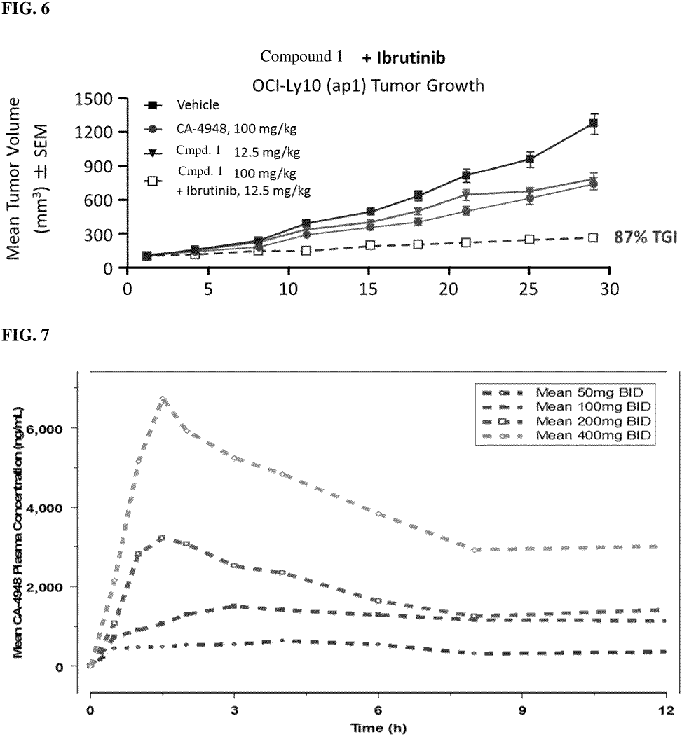

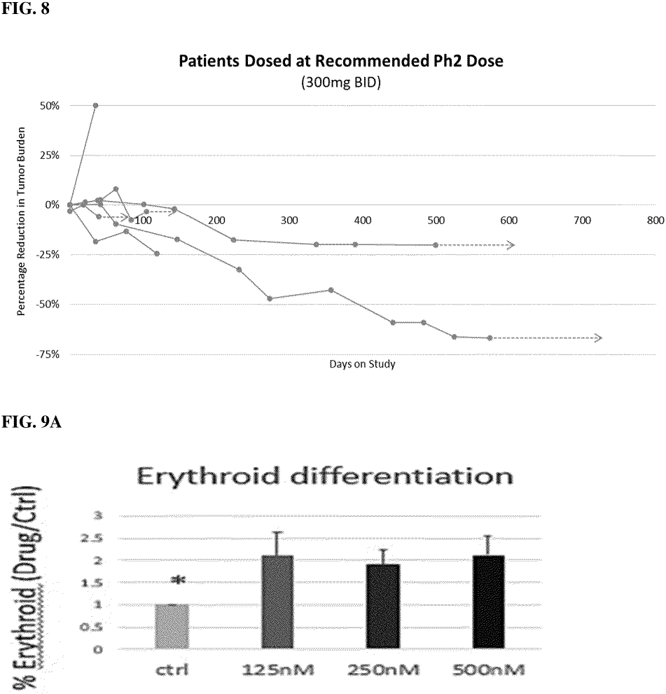

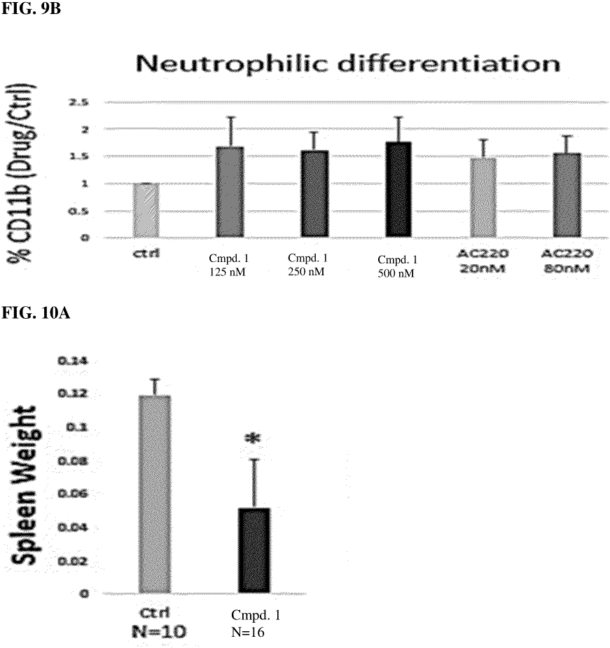

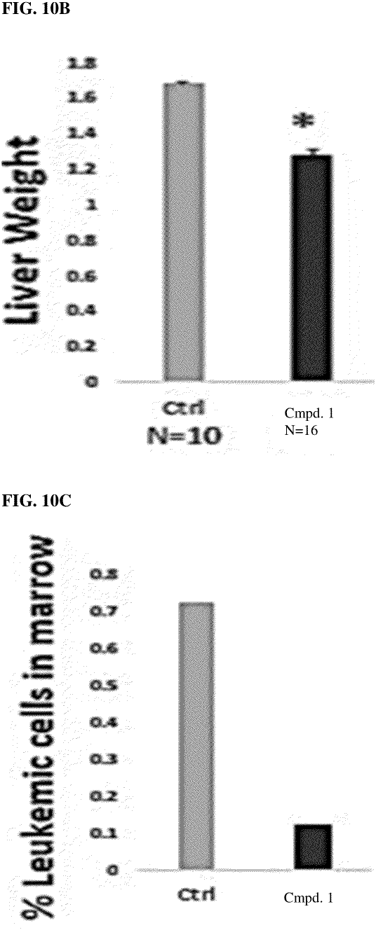

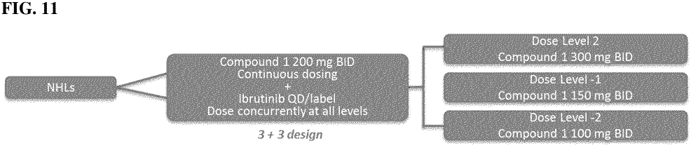

Compound 1 was well tolerated. Eight patients were exposed at the highest dose level of 400 mg BID: 2 of 5 DLT-evaluable patients had Grade 3 rhabdomyolysis (DLTs), without complications and reversible after treatment interruption and hydration / analgesic treatment - both subsequently continued treatment at lower doses of 200 or 300 mg BID, respectively. Six patients have tolerated 300 mg BID well without DLT. Most non-hematologic TEAEs were Grade 1 or 2 and manageable, including diarrhea, vomiting, fatigue, dyspnea, and myalgia. Mild/moderate, neutropenia, anemia, thrombocytopenia; only 4 Grade 3 combined episodes in 18 patients at dose levels ranging between 200 and 400 mg BID without complications (Table 2). No toxic deaths. Pharmacokinetics has shown favorable characteristics with dose-proportional increases in exposure. Similar pharmacodynamic changes were shown in cytokine reductions. Treatment duration ranged between < 1 and 18+ months with sustained disease control. Eight of 28 evaluable patients experienced overall tumor burden decreases of ≥20% from baseline – more at higher doses (Table 4). A WM patient with sustained PR underwent intra-patient dose escalation and had a dose/response relationship and very good treatment tolerance (FIG. 3). Downstream pharmacodynamic markers of IRAK4 and molecular characteristics including cell-of-origin will be presented.

TABLE 2

Summary of Adverse Effects

| Adverse Reaction | | 200 mg BID (n=5) | | 300 mg BID (n=6) (%) | | 400 mg BID (n=8) (%) |
|---|---|---|---|---|---|---|
| | All Grades (%) | Grade 3 or4 (%) | All Grades (%) | Grade 3 or 4 (%) | All Grades (%) | Grade 3 or 4 (%) |
| Diarrhea | 20 | 0 | 40 | 0 | 25 | 0 |
| Nausea | 20 | 0 | 17 | 0 | 50 | 0 |
| Vomiting | 20 | 0 | 20 | 20 | 25 | 0 |
| Constipation | 20 | 0 | 0 | 0 | 20 | 0 |
| Upper respiratory infection | 40 | 20 | 0 | 0 | 12.5 | 0 |
| Cough | 40 | 0 | 0 | 0 | 0 | 0 |
| Myalgia | 40 | 0 | 0 | 0 | 37.5 | 0 |
| Fatigue | 40 | 0 | 0 | 0 | 50 | 0 |
| Oedema | 25 | 0 | 0 | 0 | 0 | 0 |
| Headache | 20 | 0 | 0 | 0 | 12.5 | 0 |
| Dizziness | 0 | 0 | 0 | 0 | 25 | 0 |
| Insomnia | 20 | 0 | 0 | 0 | 12.5 | 0 |
| Dyspnoe | 20 | 0 | 0 | 0 | 12.5 | 12.5 |
| Peripheral sensory neuropathy | 0 | 0 | 0 | 0 | 25 | 0 |
| Back pain | 20 | 0 | 0 | 0 | 12.5 | 0 |
| Muscle weakness | 20 | 20 | 0 | 0 | 12.5 | 0 |
| Rhabdomyolysis | 0 | 0 | 0 | 0 | 25 | 25 |
| Dehydration | 20 | 0 | 0 | 0 | 12.5 | 0 |

TABLE 3

Summary of Adverse Hematological Effects

| | Compound 1 200 mg BID N=5 | | Compound 1 300 mg BID N=6 | | Compound 1 400 mg BID N=8 | |
|---|---|---|---|---|---|---|
| | All Grades n (%) [E] | Grade 3 or Higher n (%) [E] | All Grades n (%) [E] | Grade 3 or Higher n (%) [E] | All Grades n (%) [E] | Grade 3 or Higher n (%) [E] |
| Neutropenia | 2 (40.0) [3] | 2 (40.0) [2] | 1 (16.7) [5] | 1 (16.7) [1] | 2 (25.0) [3] | 0 |
| Anemia | 1 (20.0) [1] | 0 | 2 (33.3) [3] | 0 | 1 (12.5) [1] | 1 (12.5) [1] |
| Thrombocytopenia | 0 | 0 | 0 | 0 | 1 (12.5) [2] | 1 (12.5) [2] |

TABLE 4

Response Summary

| NHL | Initial Dose Level | Final Dose Level | Tumor Reduction |
|---|---|---|---|
| Subtype | | | |
| DLBCL | 50 mg qd | 50 mg qd | 24% |
| (transformed MZL) | | | |
| FL | 50 mg qd | 50 mg qd | 35% |
| FL | 100 mg qd | 50 mg qd | 49% |
| WM | 50 mg bid | 300 mg bid * | 66% (PR) |
| DLBCL | 50 mg bid | 50 mg bid | 11% |
| LPL | 200 mg bid | 300 mg bid * | 20% |
| DLBCL-GCB | 200 mg bid | 300 mg bid | 35% |
| DLBCL-ABC | 400 mg bid | 400 mg bid | 42% |
| MZL | 400 mg bid | 400 mg bid | 5% |
| MALT/MZL | 400 mg bid | 300 mg bid* | 25% |

In summary, Compound 1 demonstrated good safety and tolerance, desirable pharmacokinetic properties, and preliminary clinical activity.

### Example 3: Performance of Compound 1 in AML and MDS

### Study Design and Methods

This is a single-arm dose escalation Phase 1 study of orally administered Compound 1 monotherapy in adult patients with AML or high risk MDS (NCT04278768). This study will be conducted in 2 parts: an initial dose escalation and dose expansion phase. The starting dose level is 200 mg BID which was determined to be safe, capable of achieving relevant levels of drug exposure as well as demonstrating signs of biologic activity and clinical efficacy in an NHL Study. Three patients with AML or MDS will be enrolled at the designated dose. If none of the first 3 patients experience a DLT during the first cycle, patients may be enrolled into the next higher dose level of 300 mg bid until a safe and effective RP2D is established.

This study is expected to enroll approximately 18 patients to establish the initial RP2D. The safety population will include all patients in the study who received any dose of Compound 1, and the efficacy population will include patients who have a valid baseline and post-baseline disease assessment and received at least one dose of the study drug. Each treatment cycle of Compound 1 will be 28 days in length and repeated in the absence of toxicity or disease progression.

The major study inclusion and exclusion criteria are as follows: Relapsed or refractory AML (primary or secondary, including treatment-related) after at least one standard treatment (including chemotherapy, re-induction therapy or stem cell transplantation) based on the assessment of the investigator or high/very high risk relapsed/refractory MDS (IPSS-R criteria), following at least 6 cycles of hypomethylating agents [HMA] or evidence of early progression. Patients diagnosed with acute promyelocytic leukemia (APL, M3), blast phase of CML, allogeneic hematopoietic stem cell transplant (Allo-HSCT) within 60 days of the first dose of Compound 1 or clinically significant graft-versus-host disease (GVHD) requiring ongoing up-titration of immunosuppressive medications prior to start of Compound 1 are excluded.

The primary objective is to determine the maximum tolerated dose (MTD) and recommended Phase 2 dose (RP2D) for Compound 1 in patients with AML and high risk MDS based on the safety and tolerability, DLTs and PK/PD findings.

### Results

All initial patients completed cycle 1 with marrow blast reduction, including several marrow complete responses.

### Cohort 1 (200 Mg BID; Cycle Duration 4 Weeks)

3 patients with hr-MDS; all with ongoing treatment (currently 2-4 cycles). No DLT 1st cycle. 1 dose reduction C2 for Gr. 3 dizziness.

### Cohort 2 (300 Mg BID)

4 patients (3 AML, 1 hr-MDS), all with ongoing treatment (currently 1-2 cycles). No DLT in first 3 patients).

### Cohort 3 Open (400 Mg BID)

Open for enrollment.

### Example 4: Performance of Compound 1 in Relapsed or Refractory Hematologic Malignancies

### Study Design and Methods

This is a trial of orally administered Compound 1 in combination with ibrutinib in adult patients with relapsed or refractory hematologic malignancies. (NCT03328078). It has 2 parts: an initial dose escalation phase (Part A2) and an expansion part of 4 cohorts. In a 3×3 dose-escalation design, the starting oral dose of Compound 1 will be 200 mg BID, administered daily. Concurrently, patients receive ibrutinib daily at the labeled dose for the respective NHL subtype (560 mg or 420 mg). If well tolerated, the Compound 1 dose will be escalated to 300 mg BID. Objectives include safety/tolerance, pharmacokinetics, preliminary efficacy assessment, and exploratory biomarker correlations. Once the recommended Phase 2 dose (RP2D) for combination dose has been determined, the expansion phase (Part B) will assess efficacy (CR / ORR rate/duration), safety/tolerance, population PK, and biomarker correlations of the Compound 1 and ibrutinib combination. Part B will comprise of four cohorts which includes: 1 – MZL, 2 – DLBCL, 3 – CNSL, and 4 – NHL with adaptive ibrutinib resistance (basket design).

Cohorts 1-3 must be BTK-inhibitor naïve. The latter population will have received and responded to ibrutinib monotherapy (no primary resistance). Once they have developed adaptive, secondary resistance and shown tumor progression, the combination of ibrutinib and Compound 1 will be given. (A brief gap of ibrutinib therapy of <3 weeks is acceptable.) This cohort will include patients with ibrutinib approved or NCCN recommended indications: MCL, MZL, CLL/SLL, WM/LPL, PCNSL (NCCN-listed).

Primary objective: Preliminary efficacy signal identification of improved objective responses in cohorts 1-3 compared to historical data, and demonstration of resistance reversal in cohort 4 for by showing objective responses after preceding progression.

The estimated sample size of up to approximately 18 patients in Part A2 is based on the standard 3+3 study design for dose escalation. The exact number of patients will be determined by the number of cohorts required to establish the maximum tolerate dose (MTD) and Recommended Phase 2 Dose (RP2D) for Compound 1 when administered in combination with ibrutinib. For Part B dose expansion, up to 46 patients will be enrolled in each of 4 NHL cohorts. The safety population will include all patients in the study who received any dose of Compound 1 in combination with ibrutinib, and the efficacy population will include patients who have a valid baseline and post-baseline disease assessment and received at least one dose of the study combination drugs. Safety observations and measurements include drug exposure, AEs, safety laboratory tests, vital signs, physical examinations, ECGs, and ECOG performance status. Each treatment cycle of Compound 1 will be 21 days in length and repeated in the absence of toxicity or tumor progression and ibrutinib will be dose as per the label.

The major study inclusion and exclusion criteria for Part A2 of the combination therapy dose escalation are as follows: Diagnosis of histopathologically confirmed B-cell NHL, as per the WHO 2016 classification. Eligible NHL subtypes include follicular lymphoma, MZL, mantle cell lymphoma, DLBCL (including extranodal lymphomas of leg-, testicular-, or NOS type), CLL/SLL, primary or secondary CNS lymphoma and Waldenström macroglobulinemia / LPL. Patients with mantle cell lymphoma, MZL, WM/LPL, or CLL/SLL should meet clinical criteria for requiring treatment of their disease. Patients with the presence of an acute or chronic toxicity resulting from prior anti-cancer therapy, with the exception of alopecia, that has not resolved to Grade ≤ 1, as determined by NCI CTCAE v 4.03 within 7 days prior to start of study will be excluded.

The study treatment, endpoints are to determine the safety and tolerability, DLTs, MTD, and RP2D of oral Compound 1 in combination with ibrutinib, with secondary endpoints to assess objective response rate, (ORR), duration response rate (DOR) DCR, PFS, and OS following treatment with Compound 1 in combination with ibrutinib.

### Methods

Part A: Dose escalation monotherapy, results from the phase 1 study were presented at American Society of Hematology (ASH) 2020.

Part A2 Combination: Dose escalation, a truncated 3+3 design. Primary objectives: Safety/tolerance, maximum tolerated dose (MTD), recommended phase 2 dose (RP2D). Secondary objectives: pharmacokinetics (PK) and preliminary efficacy. Exploratory: Correlations of MYD88-L265P mutant pathway signaling and NFκB activity.

Part B Combination: A basket design of 4 expansion cohorts with a Simon 2-Stage approach, applied to each cohort.

Primary Objective: Efficacy; complete response (CR), objective response rate (ORR) and duration of response (DOR). Secondary Objectives: Safety and tolerance, progression free survival (PFS) and population-PK. Exploratory Objective: biomarkers correlations.

Inclusion criteria for Part A2, NHL subtypes (WHO 2016): Follicular lymphoma, marginal zone lymphoma (MZL), mantel cell lymphoma (MCL), diffused large B-cell lymphoma (DLBCL), chronic lymphocytic leukemia (CLL) and or small lymphocytic lymphoma (SLL), primary or secondary central nervous system lymphoma (CNS), Waldenström macroglobulinemia (WM) and lymphoplasmacytic lymphoma (LPL). Part B cohorts 1-3 include patients of similar histologies but all BTK-inhibitor naïve and for the 4th cohort, inclusion of patients with adaptive ibrutinib resistance.

Exclusions for both Parts A2 and B: Significant acute or chronic unresolved toxicity from prior therapy; serious co-morbidities.

### Example 5: Performance of Compound 1 in Autoimmune Disorders

A subject suffering from an autoimmune condition (e.g., graft vs host disease) will be administered Compound 1 in a dose escalation study starting at 50 mg. The efficacy of Compound 1 will be determined by methods known to one of ordinary skill in the art.

### Example 6: Exemplary Formulations of Compound 1

| Component | Amount per 50 mg tablet (mg) | Function |
|---|---|---|
| Intra-granular component | | |
| Compound 1 | 50.00 | Active Ingredient |
| Lactose Monohydrate (Pharmatose 200 M) | 190.00 | Diluent |
| Microcrystalline Cellulose (Avicel PH 101) | 158.20 | Diluent |
| Croscarmellose Sodium (Ac-Di-Sol) | 10.40 | Disintegrant |
| Hypromellose (HPMC E5 LV Premium) | 20.80 | Binder |
| Sodium Lauryl Sulphate (Kolliphor SLS Fine) | 5.00 | Surfactant/Solubility enhancer |
| Purified Water | q.s | Solvent |
| Extra-granular component | | |
| Croscarmellose Sodium (Ac-Di-Sol) | 10.40 | Disintegrant |
| Microcrystalline Cellulose (Avicel PH 102) | 50.00 | Diluent |
| Magnesium Stearate (Vegetable grade) | 5.20 | Lubricant |
| Core tablet weight | 500.00 | |
| Opadry AMB II 88A520004 Yellow Qualitative Composition (Polyvinyl Alcohol, Talc, Titanium Dioxide, Yellow Iron oxide, Non-irradiated, Glyceryl monocaprylocaprate, Sodium Lauryl Sulphate) | 20.00 | Coating Material |
| Purified Water | q.s | Solvent |
| Coated tablet weight | 520.00 | |
| Compound 1 drug substance | 25.00^{∗} | 10%^{∗} |
| Lactose Monohydrate (Pharmatose 200 M) | 95.00 | 38% |
| Microcrystalline Cellulose (Avicel PH 101) | 79.10^{#} | 32%^{#} |
| Croscarmellose Sodium (Ac-Di-Sol) | 5.20 | 2% |
| Hypromellose (HPMC E5 LV Premium) | 10.40 | 4% |
| Sodium Lauryl Sulphate (Kolliphor SLS Fine) | 2.50 | 1% |
| Purified Water | N/A^{$} | N/A^{$} |
| Croscarmellose Sodium (Ac-Di-Sol) | 5.20 | 2% |
| Microcrystalline Cellulose (Avicel PH 102) | 25.00 | 10% |
| Magnesium Stearate (Vegetable grade) | 2.60 | 1% |
| Core tablet weight | 250.00 | |
| Opadry AMB II 88A180040 White | 10.00 | 4% |
| Purified Water | N/A^{$} | N/A^{$} |
| Coated tablet weight | 260.00 | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating a disease or disorder in a subject in need thereof, comprising administering 25 - 500 mg of a compound to the subject, wherein the compound is 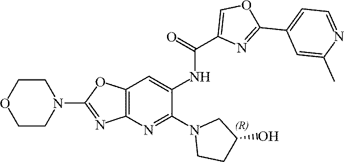 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

2. A method of treating a disease or disorder associated with IRAK4 in a subject in need thereof, comprising administering 50 - 500 mg of a compound to the subject, wherein the compound is 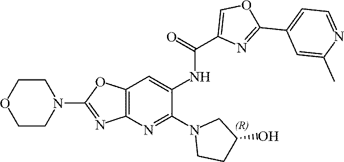 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

3. A method of treating a disease or disorder in a subject in need thereof, comprising conjointly administering 25 - 500 mg of a compound and a BTK inhibitor or a BCL-2 inhibitor to the subject, wherein the compound is 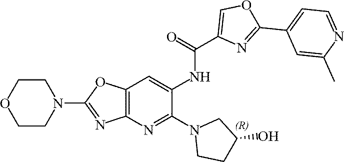 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

4. A method of treating a disease or disorder associated with IRAK4 in a subject in need thereof, comprising administering 25 - 500 mg of a compound and a BTK inhibitor or a BCL-2 inhibitor to the subject, wherein the compound is 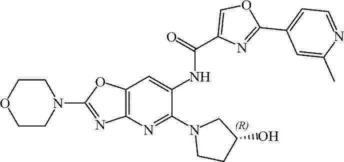 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

5. The method of claim 3 or 4, wherein the method comprises conjointly administering 25 -500 mg of a compound and BCL-2 inhibitor to the subject, wherein the compound is 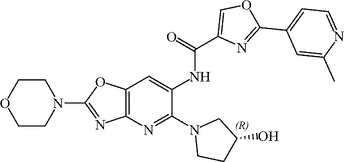 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

6. The method of claim 5, wherein the BCL-2 inhibitor is ventoclax.

7. The method of claim 5, comprising administering 400 mg of venetoclax daily.

8. The method of claim 6 or 7, wherein the ventoclax is administered orally.

9. The method of claim 5, comprising orally administering 400 mg of venetoclax daily.

10. The method of claim 2 or 3, wherein the method comprises conjointly administering 25 -500 mg of a compound and BTK inhibitor to the subject, wherein the compound is 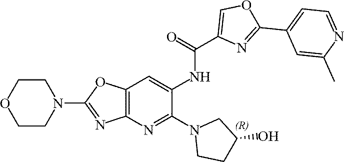 [SMILES: Cc1cc(-c2nc(C(=O)Nc3cc4oc(N5CCOCC5)nc4nc3N3CC[C@@H](O)C3)co2)ccn1]
(i.e., Compound 1) or a pharmaceutically acceptable salt thereof.

11. The method of claim 10, wherein the BTK inhibitor is ibrutinib, acalabrutinib, zanubrutinib, evobrutinib, ONO-4059, spebrutinib, or HM7 1224.

12. The method of claim 10, wherein the BTK inhibitor is ibrutinib or acalabrutinib.

13. The method of claims 10 or 11, wherein the BTK inhibitor is acalabrutinib.

14. The method of claim 13, comprising administering 200 mg of acalabrutinib daily.

15. The method claim 13 or 14, wherein the acalabrutinib is administered orally.

16. The method of claim 13, comprising orally administering 200 mg of acalabrutinib daily.

17. The method of claims 10 or 11, wherein the BTK inhibitor is ibrutinib.

18. The method of claim 17, comprising administering 420 mg of ibrutinib daily.

19. The method of claim 17, comprising administering 560 mg of ibrutinib daily.

20. The method of any one of claims 17-19, wherein the ibrutinib is administered orally.

21. The method of claim 17, comprising orally administering 420 mg of ibrutinib daily.

22. The method of claim 17, comprising orally administering 560 mg of ibrutinib daily.

23. The method of claims 10 or 11, wherein the BTK inhibitor is zanubrutinib.

24. The method of claim 23, comprising administering 160 mg of zanubrutinib twice daily.

25. The method of claim 23, comprising administering 320 mg of zanubrutinib once daily.

26. The method of claim any one of claims 21-25, wherein the zanubrutinib is administered orally.

27. The method of claim 23, comprising orally administering 160 mg of zanubrutinib twice daily.

28. The method of claim 23, comprising orally administering 320 mg of zanubrutinib once daily.

29. The method of any one of claims 1-28, wherein the compound is 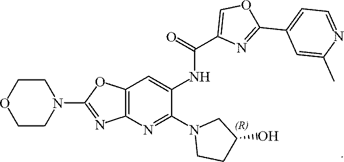
.

30. The method of any one of claims 1-28, wherein the compound is a pharmaceutically acceptable salt of 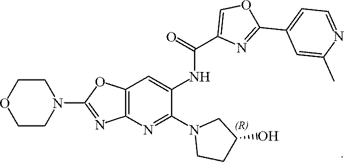
.

31. The method of any one of claims 1-30, comprising administering 100 - 400 mg of the compound to the subject twice per day.

32. The method of any one of claims 1-30, comprising administering 200 - 400 mg of the compound to the subject twice per day.

33. The method of any one of claims 1-30, comprising administering 250 - 350 mg of the compound to the subject twice per day.

34. The method of any one of claims 1-30, comprising administering about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound to the subject twice per day.

35. The method of any one of claims 1-30, comprising administering about 50 mg, about 75 mg, about 100 mg, about 200 mg, about 225 mg, about 250 mg, about 275 mg, about 300 mg, about 325 mg, about 350 mg, about 375 mg, or about 400 mg of the compound to the subject twice per day.

36. The method of any one of claims 1-30, comprising administering about 50 mg, about 100 mg, about 200 mg, or about 300 mg of the compound to the subject twice per day.

37. The method of any one of claims 1-30, comprising administering about 200 mg of the compound to the subject twice per day.

38. The method of any one of claims 1-30, comprising administering about 225 mg of the compound to the subject twice per day.

39. The method of any one of claims 1-30, comprising administering about 250 mg of the compound to the subject twice per day.

40. The method of any one of claims 1-30, comprising administering about 275 mg of the compound to the subject twice per day.

41. The method of any one of claims 1-30, comprising administering about 300 mg of the compound to the subject twice per day.

42. The method of any one of claims 1-30, comprising administering about 325 mg of the compound to the subject twice per day.

43. The method of any one of claims 1-30, comprising administering about 350 mg of the compound to the subject twice per day.

44. The method of any one of claims 1-30, comprising administering about 375 mg of the compound to the subject twice per day.

45. The method of any one of claims 1-30, comprising administering about 400 mg of the compound to the subject twice per day.

46. The method of any one of claims 1-30, comprising administering about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound to the subject once per day.

47. The method of any one of claims 1-30, comprising administering about 50 mg of the compound to the subject once per day.

48. The method of any one of claims 1-30, comprising administering about 75 mg of the compound to the subject once per day.

49. The method of any one of claims 1-30, comprising administering about 100 mg of the compound to the subject once per day.

50. The method of any one of claims 1-30, comprising administering about 125 mg of the compound to the subject once per day.

51. The method of any one of claims 1-30, comprising administering about 150 mg of the compound to the subject once per day.

52. The method of any one of claims 1-51, wherein the compound is orally administered to the subject.

53. The method of any one of claims 1-30, comprising orally administering about 200 mg of the compound to the subject twice per day.

54. The method of any one of claims 1-30, comprising orally administering about 225 mg of the compound to the subject twice per day.

55. The method of any one of claims 1-30, comprising orally administering about 250 mg of the compound to the subject twice per day.

56. The method of any one of claims 1-30, comprising orally administering about 275 mg of the compound to the subject twice per day.

57. The method of any one of claims 1-30, comprising orally administering about 300 mg of the compound to the subject twice per day.

58. The method of any one of claims 1-30, comprising orally administering about 325 mg of the compound to the subject twice per day.

59. The method of any one of claims 1-30, comprising orally administering about 350 mg of the compound to the subject twice per day.

60. The method of any one of claims 1-30, comprising orally administering about 375 mg of the compound to the subject twice per day.

61. The method of any one of claims 1-30, comprising orally administering about 400 mg of the compound to the subject twice per day.

62. The method of any one of claims 1-30, comprising administering about 50 mg of the compound to the subject once per day.

63. The method of any one of claims 1-30, comprising administering about 75 mg of the compound to the subject once per day.

64. The method of any one of claims 1-30, comprising administering about 100 mg of the compound to the subject once per day.

65. The method of any one of claims 1-30, comprising administering about 125 mg of the compound to the subject once per day.

66. The method of any one of claims 1-30, comprising administering about 150 mg of the compound to the subject once per day.

67. The method of any one of claims 1-66, wherein the disease or disorder is a cancer.

68. The method of any one of claims 1-66, wherein the disease or disorder is a hematological malignancy.

69. The method of claim 68, wherein the hematological malignancy is a non-Hodgkin’s lymphoma.

70. The method of claim 68, wherein the hematological malignancy is a leukemia or lymphoma.

71. The method of any one of claims 68-70, wherein the is hematological malignancy is myelogenous leukemia, myeloid leukemia (e.g., acute myeloid leukemia), myelodysplastic syndrome, lymphoblastic leukemia (e.g., acute lymphoblastic leukemia), chronic lymphocytic leukemia (CLL), small lymphocytic lymphoma (SLL), high risk CLL, follicular lymphoma, diffuse large B-cell lymphoma (DLBCL) (e.g., DLBCL or ABC-DLBLC), mantle cell lymphoma (MCL), Waldenstrom’s macroglobulinemia (WM), multiple myeloma, marginal zone lymphoma (MZL), Burkitt’s lymphoma, non-Burkitt high grade B cell lymphoma, extranodal marginal zone B cell lymphoma, transformed high grade B-cell lymphoma (HGBL), lymphoplasmacytic lymphoma (LPL), central nervous system lymphoma (CNSL), or MALT lymphoma.

72. The method of claim 68, wherein the hematological malignancy is myelogenous leukemia.

73. The method of claim 68, wherein the hematological malignancy is myeloid leukemia (e.g., acute myeloid leukemia).

74. The method of claim 68, wherein the hematological malignancy is acute myeloid leukemia (e.g., AML).

75. The method of claim 74, wherein the AML is primary AML.

76. The method of claim 74, wherein the AML is secondary AML.

77. The method of any one of claims 74-76, wherein the AML is treatment related AML.

78. The method of claim 68, wherein the hematological malignancy is myelodysplastic syndrome.

79. The method of claim 78, wherein the myelodysplastic syndrome is high grade.

80. The method of claim 78, wherein the myelodysplastic syndrome is low grade.

81. The method of any one of claims 78-80, wherein the myelodysplastic syndrome is high risk.

82. The method of claim 68, wherein the hematological malignancy is lymphoblastic leukemia (e.g., acute lymphoblastic leukemia).

83. The method of claim 68, wherein the hematological malignancy is chronic lymphocytic leukemia (CLL).

84. The method of claim 83, wherein the CLL is high risk CLL.

85. The method of claim 68, wherein the hematological malignancy is small lymphocytic lymphoma (SLL).

86. The method of claim 68, wherein the hematological malignancy is follicular lymphoma.

87. The method of claim 68, wherein the hematological malignancy is diffuse large B-cell lymphoma (DLBCL).

88. The method of claim 68, wherein the hematological malignancy is activated B cell-like (ABC) DLBCL.

89. The method of claim 68, wherein the hematological malignancy is germinal center B cell-like (GCB) DLBCL.

90. The method of any one of claims 87-89, wherein the DLBCL is extranodal.

91. The method of any one of claims 87-90, wherein the DLBCL is extranodal leg lymphoma, extranodal testicle lymphoma, or extra nodal not otherwise specified (NOS) type lymphoma.

92. The method of claim 68, wherein the hematological malignancy is mantle cell lymphoma.

93. The method of claim 68, wherein the hematological malignancy is Waldenstrom’s macroglobulinemia.

94. The method of claim 68, wherein the hematological malignancy is multiple myeloma.

95. The method of claim 68, wherein the hematological malignancy is marginal zone lymphoma.

96. The method of claim 68, wherein the hematological malignancy is Burkitt’s lymphoma.

97. The method of claim 68, wherein the hematological malignancy is non-Burkitt high grade B cell lymphoma.

98. The method of claim 68, wherein the hematological malignancy is extranodal marginal zone B cell lymphoma.

99. The method of claim 68, wherein the hematological malignancy is transformed high grade B-cell lymphoma (HGBL).

100. The method of claim 68, wherein the hematological malignancy is lymphoplasmacytic lymphoma (LPL).

101. The method of claim 68, wherein the hematological malignancy is CNS lymphoma.

102. The method of claim 101, wherein the CNS lymphoma is primary CNS lymphoma (PCNSL).

103. The method of claim 68, wherein the hematological malignancy is MALT lymphoma.

104. The method of any one of claims 68-103, wherein the hematological malignancy is relapsed.

105. The method of any one of claims 68-103, wherein the hematological malignancy is refractory.

106. The method of claim 67, wherein the cancer is selected from brain cancer, kidney cancer, liver cancer, stomach cancer, penile cancer, vaginal cancer, ovarian cancer, gastric cancer, breast cancer, bladder cancer, colon cancer, prostate cancer, pancreatic cancer, lung cancer, cervical cancer, epidermal cancer, prostate cancer, head or neck cancer.

107. The method of claim 106, wherein the cancer is pancreatic cancer.

108. The method of claim 106, wherein the cancer is colon cancer.

109. The method of any one of claims 106-108, wherein the cancer is a solid tumor.

110. The method of any one of claims 106-108, wherein the cancer is relapsed.

111. The method of any one of claims 106-108, wherein the cancer is refractory.

112. The method of any one of claims 1-111, wherein the disease or disorder is resistant to treatment with a BTK inhibitor.

113. The method of claim 112, wherein the disease or disorder is resistant to treatment with ibrutinib, acalabrutinib, zanubrutinib, evobrutinib, ONO-4059, spebrutinib, or HM7 1224.

114. The method of claim 112, wherein the disease or disorder is resistant to treatment with ibrutinib.

115. The method of claim 112, wherein the disease or disorder is resistant to treatment with acalabrutinib.

116. The method of any one of claims 1-66, wherein the disease or disorder is an inflammatory disease or disorder.

117. The method of claim 116, wherein the inflammatory disease or disorder is an autoimmune disease or disorder.

118. The method of claim 116, wherein the inflammatory disease or disorder is an ocular allergy, conjunctivitis, keratoconjunctivitis sicca, vernal conjunctivitis, allergic rhinitis, autoimmune hematological disorders, hemolytic anemia, aplastic anemia, pure red cell anemia, idiopathic thrombocytopenia, systemic lupus erythematosus, rheumatoid arthritis, polychondritis, scleroderma, Wegener granulamatosis, dermatomyositis, chronic active hepatitis, myasthenia gravis, Steven- Johnson syndrome, idiopathic sprue, autoimmune inflammatory bowel disease, ulcerative colitis, Crohn’s disease, irritable bowel syndrome, celiac disease, periodontitis, hyaline membrane disease, kidney disease, glomerular disease, alcoholic liver disease, multiple sclerosis, endocrine opthalmopathy, Grave’s disease, sarcoidosis, alveolitis, chronic hypersensitivity pneumonitis, primary biliary cirrhosis, uveitis (anterior or posterior), Sjogren’s syndrome, interstitial lung fibrosis, psoriatic arthritis, systemic juvenile idiopathic arthritis, nephritis, vasculitis, diverticulitis, interstitial cystitis, glomerulonephritis, idiopathic nephrotic syndrome, minimal change nephropathy, chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic urticarial dysplasia, Behcet’s disease, incontinentia pigmenti, Paget’s disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison’s disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, 63rticaria, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, 63rticarial, bullous pemphigoid, pemphigus vulgaris, pemphigus foliaceus, paraneoplastic pemphigus, epidermolysis bullosa acquisita, acute or chronic gout, chronic gouty arthritis, psoriasis, psoriatic arthritis, rheumatoid arthritis, Cryopyrin Associated Periodic Syndrome (CAPS) and osteoarthritis.

119. The method of claim 116 or 117, wherein the inflammatory disease or disorder is hypercytokinemia.

120. The method of claim 119, wherein the hypercytokinemia is induced by an infectious agent.

121. The method of claim 120, wherein the infectious agent is a virus.

122. The method of claim 121, wherein the virus is a coronavirus (e.g., COVID-19).

123. The method of claim 120, wherein the infectious agent is a bacteria.

124. The method of claim 116 or 117, wherein the inflammatory disease or disorder is graft vs host disease (GVHD).

125. The method of claim 116 or 117, wherein the GVHD is chronic graft vs host disease (cGVHD).

126. The method of claim 116 or 117, wherein the GVHD is sclerodermatous GVHD, steroid resistant GVHD, cyclosporin-resistant GVHD, GVHD, oral GVHD, reticular oral GVHD, erosive GVHD, or ulcerative oral GVHD.

127. The method of claim 124 or 125, wherein the GVHD is sclerodermatous GVHD.

128. The method of claim 124 or 125, wherein the GVHD is oral GVHD.

129. The method of claim 124 or 125, wherein the GVHD is reticular oral GVHD.

130. The method of claim 124 or 125, wherein the GVHD is erosive GVHD.

131. The method of claim 124 or 125, wherein the GVHD is ulcerative oral GVHD.

132. The method of any one of claims 124-131, wherein the GVHD is overlap chronic GVHD.

133. The method of any one of claims 124-132, wherein the GVHD is classic chronic GVHD.

134. The method of any one of claims 124-133, wherein the GVHD is steroid resistant GVHD.

135. The method of any one of claims 124-134, wherein the GVHD is cyclosporin-resistant GVHD.

136. The method of any one of claims 124-135, wherein the GVHD is refractory.

137. The method of any one of claims 124-136, wherein the GVHD is relapsed.

138. The method of any one of claims 1-137, wherein the disease or disorder is associated with chronic anemia.

139. The method of any one of claims 1-66, wherein the disease or disorder is chronic anemia.

140. The method of any one of claims 1-139, wherein the disease or disorder is associated with transfusion dependency.

141. The method of any one of claims 1-140, wherein the subject is an adult human.

142. The method of any one of claims 1-141, wherein the subject has previously received at least one anti-cancer therapy (e.g., an anti-cancer therapy or an anti-inflammatory therapy).

143. The method of claim 142, wherein the subject has previously received one anti-cancer therapy.

144. The method of claim 142, wherein the subject has previously received two anti-cancer therapies.

145. The method of claim 142, wherein the subject has previously received three anti-cancer therapies.

146. The method of claim 142, wherein the subject has previously received four anti-cancer therapies.

147. The method of claim 142, wherein the subject has previously received five anti-cancer therapies.

148. The method of any one of claims 142-147, wherein the at least one anti-cancer therapy is selected from an anti-CD20 antibody, a nitrogen mustard, a steroid, a purine analog, a DNA a topoisomerase inhibitor, a DNA intercalator, a tubulin inhibitor, a BCL-2 inhibitor, a proteasome inhibitor, a toll-like receptor inhibitor, a kinase inhibitor, an SRC kinase inhibitor, a PI3K kinase inhibitor, BTK inhibitor, a glutaminase inhibitor, a steroid, a PD-1 inhibitor a PD-L1 inhibitor, and a methylating agent; or a combination thereof.

149. The method of any one of claims 142-147, wherein the anti-cancer therapy is selected from ibrutinib, rituximab, bendamustine, bortezomib, dexamethasone, chlorambucil, cladribine, cyclophosphamide, doxorubicin, vincristine, venetoclax, ifosfamide, prednisone, oprozomib, ixazomib, acalabrutinib, zanubrutinib, IMO-08400, idelalisib, umbrelasib, CB-839, fludarabine, and thalidomide; or a combination thereof.

150. The method of any one of claims 142-147, wherein the therapy is dexamethasone.

151. The method of any one of claims 142-147, wherein the anti-cancer therapy is ibrutinib.

152. The method of any one of claims 142-147, wherein the anti-cancer therapy is ibrutinib and rituximab.

153. The method of any one of claims 142-147, wherein the anti-cancer therapy is bendamustine.

154. The method of any one of claims 142-147, wherein the anti-cancer therapy is bendamustine and rituximab.

155. The method of any one of claims 142-147, wherein the anti-cancer therapy is bortezomib.

156. The method of any one of claims 142-147, wherein the anti-cancer therapy is bortezomib and dexamethasone.

157. The method of any one of claims 142-147, wherein the anti-cancer therapy is bortezomib and rituximab.

158. The method of any one of claims 142-147, wherein the anti-cancer therapy is bortezomib, rituximab, and dexamethasone.

159. The method of any one of claims 142-147, wherein the anti-cancer therapy is chlorambucil.

160. The method of any one of claims 142-147, wherein the anti-cancer therapy is cladribine.

161. The method of any one of claims 142-147, wherein the anti-cancer therapy is cladribine and rituximab.

162. The method of any one of claims 142-147, wherein the anti-cancer therapy is cyclophosphamide, doxorubicin, vincristine, prednisone, and rituximab (i.e., CHOP-R).

163. The method of any one of claims 142-147, wherein the anti-cancer therapy is cyclophosphamide, prednisone, and rituximab (i.e., CPR).

164. The method of any one of claims 142-147, wherein the anti-cancer therapy is fludarabine.

165. The method of any one of claims 142-147, wherein the anti-cancer therapy is fludarabine and rituximab.

166. The method of any one of claims 142-147, wherein the anti-cancer therapy is fludarabine, cyclophosphamide, and rituximab.

167. The method of any one of claims 142-147, wherein the anti-cancer therapy is rituximab.

168. The method of any one of claims 142-141, wherein the anti-cancer therapy is rituximab, cyclophosphamide, and dexamethasone (i.e., RCD).

169. The method of any one of claims 142-147, wherein the anti-cancer therapy is thalidomide.

170. The method of any one of claims 142-147, wherein the anti-cancer therapy is thalidomide and rituximab.

171. The method of any one of claims 142-147, wherein the anti-cancer therapy is venetoclax.

172. The method of any one of claims 142-147, wherein the anti-cancer therapy is cyclophosphamide, bortezomib, and dexamethasone (i.e., R-CyBorD).

173. The method of any one of claims 142-147, wherein the anti-cancer therapy a hypomethylating agent.

174. The method of any one of claims 142-147, wherein the subject has previously received at least 6 cycles of a hypomethylating agent.

175. The method of any one of claims 1-174, wherein the subject has previously received etoposide chemo-mobilization therapy.

176. The method of any one of claims 1-175, wherein the subject has previously received a bone marrow transplant.

177. The method of any one of claims 1-176, wherein the subject has previously received a hematopoietic cell transplantation.

178. The method of any one of claims 1-177, wherein the subject has previously received a stem cell transplant.

179. The method of any one of claims 1-178, wherein the subject has previously received an autologous stem cell transplant.

180. The method of any one of claims 1-179, wherein the subject has previously received an allogenic stem cell transplant.

181. The method of any one of claims 1-180, wherein the subject has previously received carmustine, etoposide, cytarabine, and melphalan (i.e., BEAM conditioning).

182. The method of any one of claims 1-181, wherein the subject has previously received re-induction therapy.

183. The method of any one of claims 142-182, wherein the subject has previously achieved a partial response.

184. The method of any one of claims 142-182, wherein the subject has previously achieved a good partial response.

185. The method of any one of claims 142-182, wherein the subject has previously achieved a complete response.

186. The method of any one of claims 1-185, wherein the subject has a mutation in RICTOR.

187. The method of any one of claims 1-186, wherein the subject has a N1065S mutation in RICTOR.

188. The method of any one of claims 1-187, wherein the subject has a mutation in MYD88.

189. The method of any one of claims 1-188, wherein the subject has a L265P mutation in MYD88.

190. The method of any one of claims 1-189, wherein the subject has a mutation in TET2.

191. The method of any one of claims 1-190, wherein the subject does not have a mutation in CXCR4.

192. The method of any one of claims 1-191, wherein the subject has a mutation in CXCR4.

193. The method of any one of claims 1-192, wherein the subject shows early progression.

194. The method of any one of claims 1-193, wherein the subject has not previously received a BTK inhibitor.

195. The method of any one of claims 1-194, wherein following administration of the compound, the subject achieves a partial response.

196. The method of any one of claims 1-195, wherein following administration of the compound, the subject achieves a good partial response.

197. The method of any one of claims 1-196, wherein following administration of the compound, the subject achieves a complete response.

198. The method of any one of claims 1-197, wherein following administration of the compound, the subjects IL-1 induced signaling decreases.

199. The method of any one of claims 1-198, wherein following administration of the compound, the subject’s cytokine production decreases.

200. The method of any one of clams 1-199, wherein the compound is administered until disease progression or unacceptable toxicity.

201. A pharmaceutical composition comprising 50 - 500 mg of compound and a pharmaceutically acceptable excipient, wherein the compound is 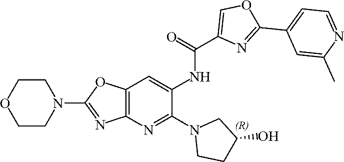 [SMILES: Cc1cc(-c2nc(C(=O)Nc3cc4oc(N5CCOCC5)nc4nc3N3CC[C@@H](O)C3)co2)ccn1]
or a pharmaceutically acceptable salt thereof.

202. The pharmaceutical composition of claim 201, wherein the compound is 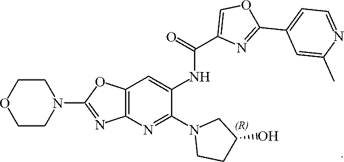
.

203. The pharmaceutical composition of claim 201, wherein the compound is a pharmaceutically acceptable salt of 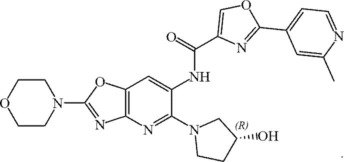
.

204. The pharmaceutical composition of any one of claims 201-203, comprising 50 - 400 mg of the compound.

205. The pharmaceutical composition of any one of claims 201-204, comprising 100 - 400 mg of the compound.

206. The pharmaceutical composition of any one of claims 201-204, comprising 200 - 400 mg of the compound.

207. The pharmaceutical composition of any one of claims 201-204, comprising 250 - 350 mg of the compound.

208. The pharmaceutical composition of any one of claims 201-204, comprising about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound.

209. The pharmaceutical composition of any one of claims 201-204, comprising about 50 mg, about 75 mg, about 100 mg, about 200 mg, about 225 mg, about 250 mg, about 275 mg, about 300 mg, about 325 mg, about 350 mg, about 375 mg, or about 400 mg of the compound.

210. The pharmaceutical composition of any one of claims 201-204, comprising about 50 mg, about 100 mg, about 200 mg, or about 300 mg of the compound.

211. The pharmaceutical composition of any one of claims 201-204, comprising about 50 mg of the compound.

212. The pharmaceutical composition of any one of claims 201-204, comprising about 200 mg of the compound.

213. The pharmaceutical composition of any one of claims 201-204, comprising about 225 mg of the compound.

214. The pharmaceutical composition of any one of claims 201-204, comprising about 250 mg of the compound.

215. The pharmaceutical composition of any one of claims 201-204, comprising about 275 mg of the compound.

216. The pharmaceutical composition of any one of claims 201-204, comprising about 300 mg of the compound.

217. The pharmaceutical composition of any one of claims 201-204, comprising about 325 mg of the compound.

218. The pharmaceutical composition of any one of claims 201-204, comprising about 350 mg of the compound.

219. The pharmaceutical composition of any one of claims 201-204, comprising about 375 mg of the compound.

220. The pharmaceutical composition of any one of claims 201-204, comprising about 400 mg of the compound.

221. The pharmaceutical composition of any one of claims 201-220, wherein the compound comprises about 5%, about 10%, or about 15% of the total weight of the pharmaceutical composition.

222. The pharmaceutical composition of any one of claims 201-221, wherein the compound comprises about 10% of the total weight of the pharmaceutical composition.

223. The pharmaceutical composition of any one of claims 201-222, wherein the pharmaceutical composition further comprises a diluent.

224. The pharmaceutical composition of claim 223, wherein the diluent comprises lactose monohydrate or microcrystalline cellulose; or a combination thereof.

225. The pharmaceutical composition of claim 223 or 224, wherein the diluent comprises lactose monohydrate and microcrystalline cellulose.

226. The pharmaceutical composition of any one of claims 223-225, wherein the diluent comprises about 75%, about 80%, or about 85% of the total weight of the pharmaceutical composition.

227. The pharmaceutical composition of any one of claims 224-226, wherein the diluent comprises about 80% of the total weight of the pharmaceutical composition.

228. The pharmaceutical composition of any one of claims 201-227, wherein the pharmaceutical composition further comprises a binder.

229. The pharmaceutical composition of claim 228, wherein the binder is hypromellose.

230. The pharmaceutical composition of claim 228 or 229, wherein the binder comprises about 2.5%, about 5%, or about 7.5% of the total weight of the pharmaceutical composition.

231. The pharmaceutical composition of any one of claims 228-230, wherein the binder comprises about 5% of the total weight of the pharmaceutical composition.

232. The pharmaceutical composition of any one of claims 201-231, wherein the pharmaceutical composition further comprises a surfactant.

233. The pharmaceutical composition of claim 232, wherein the surfactant is a lauryl sulphate (e.g., sodium lauryl sulphate).

234. The pharmaceutical composition of claim 232 or 233, wherein the surfactant comprises about 0.5%, about 1%, or about 1.5% of the total weight of the pharmaceutical composition.

235. The pharmaceutical composition of any one of claims 232-234, wherein the surfactant is about 1% of the total weight of the pharmaceutical composition.

236. The pharmaceutical composition of any one of claims 201-235, wherein the pharmaceutical composition further comprises a disintegrant.

237. The pharmaceutical composition of claim 236, wherein the disintegrant is a croscarmellose (e.g., croscarmellose sodium).

238. The pharmaceutical composition of claim 236 or 237, wherein the disintegrant comprises about 1.5%, about 2%, or about 2.5% of the total weight of the pharmaceutical composition.

239. The pharmaceutical composition of any one of claims 236-238, wherein the disintegrant comprises about 1% of the total weight of the pharmaceutical composition.

240. The pharmaceutical composition of any one of claims 201-239, wherein the pharmaceutical composition further comprises a lubricant.

241. The pharmaceutical composition of claim 240, wherein the lubricant is a stearate (e.g., magnesium stearate).

242. The pharmaceutical composition of claim 240 or 241, wherein the lubricant comprises about 0.5%, about 1%, or about 1.5% of the total weight of the pharmaceutical composition.

243. The pharmaceutical composition of any one of claims 240-242, wherein the lubricant comprises about 1% of the total weight of the pharmaceutical composition.

244. The pharmaceutical composition of any one of claims 201-243, wherein the pharmaceutically acceptable excipient is water.

245. The pharmaceutical composition of any one of claims 201-244, wherein the pharmaceutical composition is in the form of a tablet or a capsule.

246. The pharmaceutical composition of any one of claims 201-245, wherein the pharmaceutical composition is in the form of a tablet.

247. The pharmaceutical composition of claim 246, wherein the table is coated with polyvinyl alcohol, talc, titanium dioxide, non-irradiated yellow iron oxide, glyceryl monocaprylocaprate, or sodium lauryl sulphate; or a combination thereof.

248. The pharmaceutical composition of claim 246 or 247, wherein the table is coated with polyvinyl alcohol, talc, titanium dioxide, non-irradiated yellow iron oxide, glyceryl monocaprylocaprate, and sodium lauryl sulphate.

249. An integer number of unit dosage form(s) comprising the pharmaceutical composition of any one of claims 201-248, wherein the integer number of the unit dosage forms supplies the entire dose of any one of claims 1-200.

250. The unit dosage forms of claim 249, wherein 1, 2, 3, 4, 5, or 6 unit dosage forms supply the entire dose.

251. The unit dosage forms of claim 249, wherein one unit dosage form supplies the entire dose.

252. The unit dosage forms of claim 249, wherein two unit dosage forms supply the entire dose.

253. The unit dosage forms of claim 249, wherein three unit dosage forms supply the entire dose.

254. The unit dosage forms of claim 249, wherein four unit dosage forms supply the entire dose.

255. The unit dosage forms of claim 249, wherein five unit dosage forms supply the entire dose.

256. The unit dosage forms of claim 249, wherein six unit dosage forms supply the entire dose.

257. The method of any one of claims 1-200, wherein Compound 1 is administered to the subject continuously.

258. The method of any one of claims 1-200, wherein Compound 1 is administered to the subject intermittently.

259. The method of claim 258, wherein the method comprises continuous administration interrupted by one or more drug holidays.

260. The method of claim 259, wherein each drug holiday lasts for 1, 2, 3, 4, 5, 6, 7, 8, 9, 10, 11, 12, 13, or 14 days.

261. The method of claim 259 or 260, wherein each drug holiday lasts for 7 days.

262. The method of any one of claims 258-261, wherein Compound 1 is administered daily for three weeks followed by a one-week drug holiday.

263. The method of any one of claims 257-262, wherein the method continues cycling between three-week periods of administration and one-week drug holidays until a change of disease state is observed.

264. The method of claim 263, wherein the change of disease state is a complete response.

265. The method of claim 263, wherein the change of disease state is a partial response.

266. The method of claim 263, wherein the change of disease state is unacceptable toxicity.
